(19) United States
(12) Patent Application Publication
(10) Pub. No.: US 2021/0008297 A1
(43) Pub. Date: Jan. 14, 2021

(54) GASKET PRESSING TOOL FOR SYRINGE OR LIQUID MEDICINE EJECTION TOOL, AND LIQUID MEDICINE ADMINISTRATION TOOL INCLUDING THE SAME

(71) Applicant: TERUMO KABUSHIKI KAISHA, Tokyo (JP)

(72) Inventor: Hitoshi OKIHARA, Fujinomiya-shi (JP)

(73) Assignee: TERUMO KABUSHIKI KAISHA, Tokyo (JP)

(21) Appl. No.: 17/032,758

(22) Filed: Sep. 25, 2020

Related U.S. Application Data
(63) Continuation of application No. PCT/JP2019/012928, filed on Mar. 26, 2019.

(30) Foreign Application Priority Data
Mar. 28, 2018 (JP) 2018-063371
Mar. 29, 2018 (JP) 2018-066279
Mar. 29, 2018 (JP) 2018-066280

(51) Int. Cl. A61M 5/315

(52) U.S. Cl. CPC A61M 5/31586; A61B 2017/00269; A61M 2205/0216; A61M 5/31528

(57) ABSTRACT

A liquid medicine ejection tool includes a syringe and a gasket pressing tool. The gasket pressing tool includes a gasket pressing member that can enter a gasket in the syringe. The gasket has an original lumen portion axial length, and a gasket-mounting portion of the gasket pressing member has an axial length longer than the original lumen portion axial length. When the gasket pressing member is mounted on the barrel of the gasket, a mounting portion distal surface of the gasket-mounting portion abuts on a lumen distal surface of a lumen portion of the gasket, and a gasket proximal surface pressing portion of the gasket pressing member becomes close to or abuts on a proximal surface of the gasket.

### CROSS-REFERENCES TO RELATED APPLICATIONS

This application is a continuation of International Application No. PCT/JP2019/012928 filed on Mar. 26, 2019, which claims priority to Japanese Patent Application No. 2018-063371, filed on Mar. 28, 2018, Japanese Patent Application No. 2018-066279, filed on Mar. 29, 2018, and Japanese Patent Application No. 2018-066280, filed on Mar. 29, 2018, the entire content of each of which is incorporated herein by reference.

### FIELD OF THE INVENTION

The present invention generally relates to a gasket pressing tool for a syringe or a liquid medicine ejection tool, and a liquid medicine administration tool including the same.

### BACKGROUND DISCUSSION

A type of syringe known as a threaded syringe is configured to advance a plunger by a screwing operation of a screw so as to be able to discharge a relatively high-viscosity liquid medicine from the syringe barrel. Examples of such a type of injector include, for example, JP 2007-520276 A (Patent Literature 1), JP 2008-167954 A (Patent Literature 2), and JP 2005-535415 A (Patent Literature 3).
Patent Literature 1: JP 2007-520276 A (US 2007-287958 A1)
Patent Literature 2: JP 2008-167954 A
Patent Literature 3: JP 2005-535415 A (US 2003-055386 A1)

### SUMMARY

In an injector 3 of Patent Literature 1, a threaded rod 1 is attached to a plunger 6 at its distal end. The plunger 6 is located within a syringe body 4 having a proximal flange 8. A cap 5 is connected to the flange 8. A handle 2 which acts as a thumbwheel for turning the rod and advancing the plunger 6, is attached at a proximal end of the rod 1. A stopper 7 is located within the syringe body 4 to form an air-tight seal when a force of the advancing plunger is applied against a viscous substance within the syringe body. The viscous substance is ejected through the distal end of the syringe body into an appropriate receiving device.

In syringes including the injector as disclosed in Patent Literature 1, a gasket made of an elastic material is used as the above-described stopper.

Recently, endoscopic mucosal resection using local injection of a highly viscous substance has been performed. As the highly viscous substance, hyaluronic acid, which is a medicine, is generally used. As the endoscopic mucosal resection, for example, partial resection of an inner wall of a digestive tract using an endoscope is performed.

In the endoscopic mucosal resection, it is important to control the injection amount of hyaluronic acid. However, the hyaluronic acid, which is the highly viscous substance, is barely administered unless being extruded with a considerable pressure. In addition, there is a case where the hyaluronic acid is not immediately administered even after performing the administration operation.

The inventors of the present application have conducted earnest studies and found that a time lag between the administration operation and actual outflow of the highly viscous substance is caused by a deformation of a gasket. Since the gasket is elastically deformed, the highly viscous substance does not flow out until the elastic deformation is completed since the start of injection. It has been found that it is difficult to favorably perform the administration of the highly viscous substance to a target site due to this time lag.

A first object of the present disclosure is to provide a liquid medicine ejection tool and a liquid medicine administration tool including the same which can reduce a time lag between an administration operation of a liquid medicine and actual outflow of the liquid medicine and administer the liquid medicine to a target site in a timely and favorable manner even when the liquid medicine is a highly viscous substance.

In addition, in a syringe of Patent Literature 2, a threaded flange 4 having a female screw 3 is mounted on a syringe barrel 1, a male screw 7 formed on a shaft portion 6 of a plunger 5 is screwed into a female screw 3 of the threaded flange 4, and a click protrusion 9 is engaged with an axial groove 8 formed in the shaft portion of the plunger 5 every time the shaft portion 6 of the plunger 5 is rotated by a predetermined angle. The syringe includes: a movable piece 10 which is provided on the outer peripheral side of the shaft portion 6 of the plunger 5 to be movable in a radial direction and has the click protrusion 9; and a pressing piece 11 which presses and moves the movable piece 10 toward the shaft portion 6 side of the plunger 5 so that the click protrusion 9 is engaged with the axial groove 8 formed in the shaft portion 6 of the plunger 5.

Further, the threaded flange 4 has slots 16 at both ends with which the flange 2 of the syringe barrel 1 fits. The threaded flange 4 is engaged and integrated with the flange 2 by being rotated to a position overlapping the flange 2, and is detached from the flange 2 by being rotated to a position where the threaded flange 4 intersects the flange 2 around 90°.

The female screw 3 is formed at the center of the threaded flange 4, and the male screw 7 of the shaft portion 6 of the plunger 5 is screwed into the female screw 3. In the plunger 5, a gasket 18 is mounted to a distal end of the shaft portion 6, and the male screw 7 of the shaft portion 6 is screwed into the female screw 3 of the threaded flange 4. Further, the plunger 5 is configured such that ejection is performed every time the shaft portion 6 of the plunger 5 rotates by a predetermined angle by rotating the shaft portion 6 of the plunger 5 via a handle 19 in a state where the threaded flange 4 is mounted to the flange 2 of the syringe barrel 1.

When hyaluronic acid, which is a highly viscous substance, is administered using the syringe as disclosed in Patent Literature 2, the plunger 5 advances by rotating the plunger 5, the gasket 18 advances, and the highly viscous substance is discharged. The gasket 18 and the plunger 5 are pushed rearward by the resistance generated at the time of pressing the highly viscous substance.

It has been found in the course of the inventor studies that a pressing force to the rear side during pressing the above-described highly viscous substance is applied to a connecting portion that accommodates the flange of the syringe barrel to deform the connecting portion to be expanded. Furthermore, it has been found that a time lag between an administration operation and actual outflow of the highly viscous substance is caused by this deformation of the connecting portion, such that the highly viscous substance does not flow out until the deformation of the connecting portion is completed since the start of injection, and that it is difficult to favorably perform the administration of the highly viscous substance to a target site due to this time lag.

A second object of the present disclosure is to provide a gasket pressing tool for a syringe and a liquid medicine administration tool including the same which can administer a highly viscous substance to a target site in a timely and favorable manner with a short time lag between an administration operation of the highly viscous substance and actual outflow of the highly viscous substance.

In addition, a threaded syringe of Patent Literature 3 is provided with a mechanism for generating a click sound when a plunger rotates in order to provide a clinician with a tactile or audible indication for the rotation of the plunger. In this mechanism, the click sound is generated by an impact when a click piece engages with a groove provided in the plunger.

In the mechanism of Patent Literature 3, the magnitude of the generated click sound differs depending on a rotation speed of the plunger, and almost no click sound is generated when the plunger is slowly rotated.

In addition, recently, endoscopic mucosal resection using local injection of a highly viscous substance has been performed. As the highly viscous substance, hyaluronic acid, which is a medicine, is generally used. As the endoscopic mucosal resection, for example, partial resection of an inner wall of a digestive tract using an endoscope is performed. In the endoscopic mucosal resection, an endoscopic operation and injection of the highly viscous substance are performed by different operators. For this reason, it is difficult for an endoscope operator to know the injection amount of the highly viscous substance. With a medicine administration tool that generates the click sound as described above, the click sound can indicate the injection amount. In this case, however, the click sound is preferably generated only during the injection, and is required not to be generated during reverse rotation or idling, for example.

A third object of the present disclosure is to provide a gasket pressing tool for a syringe and a liquid medicine administration tool including the same which can generate a certain volume of sound every time a plunger is rotated by a predetermined amount during a medicine ejection operation and generate the sound only when pressing the gasket to move forward.

Disclosed is liquid medicine ejection tool aimed at least at the first object and provided with: a syringe that includes a barrel having a flange at a proximal end portion and a gasket slidable inside the barrel and made of an elastic material; and a gasket pressing tool used by being mounted on the syringe, wherein

the gasket includes a tubular portion, a distal closing portion that closes a distal end of the tubular portion, a lumen portion defined by the tubular portion and the distal closing portion, and an annular rib provided on an outer surface of the tubular portion,

the gasket pressing tool includes: a gasket pressing member having a gasket-mounting portion, which is capable of entering the lumen portion of the gasket and made of a material harder than the gasket, and a gasket proximal surface pressing portion provided at a proximal end portion of the gasket-mounting portion; a syringe-mounting member that movably accommodates the gasket pressing member and has a proximal barrel-mounting portion that is mountable to a proximal end portion of the barrel having the flange; and a plunger that has a distal end portion entering the syringe-mounting member and is capable of pressing the gasket pressing member in a distal direction,

the gasket has an original lumen portion axial length S when the gasket is not inserted into the barrel and the gasket pressing member is not mounted, the gasket-mounting portion of the gasket pressing member has an axial length T, and the axial length T is longer than the original lumen portion axial length S, and

when the gasket is inserted into the barrel and the gasket pressing member is mounted, a mounting portion distal surface of the gasket-mounting portion of the gasket pressing member abuts on a lumen distal surface of the lumen portion of the gasket, and the gasket proximal surface pressing portion of the gasket pressing member becomes close to or abuts on a proximal surface of the gasket.

Also disclosed is a liquid medicine administration tool including: the above-described liquid medicine ejection tool; a liquid medicine charged in the syringe; and a sealing member sealing the distal end portion of the barrel.

Disclosed is gasket pressing tool used by being mounted on a syringe and aimed at least at the second object, which includes: a barrel having an annular flange at a proximal end portion; and a gasket that is slidable in the barrel and is made of an elastic material,

the gasket pressing tool including: a gasket pressing member configured to press the gasket; a tubular syringe-mounting member that movably accommodates the gasket pressing member and is mountable on the proximal end portion of the barrel; and a plunger which has a distal end portion entering the syringe-mounting member and is capable of pressing the gasket pressing member in a distal direction,

wherein the plunger includes: the distal end portion capable of pressing the gasket pressing member; a shaft portion that extends in a proximal direction from the distal end portion and has a shaft-side screwing portion on an outer peripheral surface; and a handle configured to rotate the plunger provided at a proximal end portion of the shaft portion,

the syringe-mounting member includes: a body portion capable of accommodating the flange of the barrel; a tubular accommodation portion which extends from a proximal end of the body portion in a proximal direction of the gasket pressing tool and is capable of accommodating the gasket pressing member; and a tubular insertion portion which extends from a proximal end of the tubular accommodation portion in the proximal direction of the gasket pressing tool and in which the shaft portion of the plunger is inserted,

the insertion portion of the syringe-mounting member has an insertion-portion-side screwing portion, which is screwed with the shaft-side screwing portion of the shaft portion, on an inner peripheral surface,

the body portion of the syringe-mounting member includes: a distal-side support portion capable of supporting a distal surface of the flange; a proximal-side support portion capable of supporting a proximal surface of the flange; a connecting portion connecting the distal-side support portion and the proximal-side support portion; a flange accommodation portion which is formed of the distal-side support portion, the proximal-side support portion, and the connecting portion, and is capable of accommodating the flange; a flange insertion opening which is open to a lateral side of the body portion and is configured to insert the flange into the flange accommodation portion, and

the distal-side support portion has an axial thickness of 5 to 25 mm along a central axis of the syringe-mounting member, the proximal-side support portion has an axial thickness of 5 to 25 mm along the central axis of the syringe-mounting member, and the connecting portion has a radial width of 1 to 5 mm along a radial direction of the syringe-mounting member.

Also disclosed is a liquid medicine administration tool including: the above-described gasket pressing tool; the syringe; a liquid medicine charged in the syringe; and a sealing member sealing the distal end portion of the barrel.

Disclosed is a gasket pressing tool for a syringe, which is used by being mounted on a syringe having a barrel and a gasket slidable in the barrel and is aimed at least at the third object,

the gasket pressing tool including: a gasket pressing assembly which includes a gasket pressing member and a sound generating member; a syringe-mounting member which has a syringe-mounting portion and movably accommodates the gasket pressing assembly; and a plunger which enters the syringe-mounting member on a distal side and is capable of pressing the gasket pressing assembly by rotation,

wherein the sound generating member includes a claw portion which is elastically deformable, the gasket pressing member includes a wall portion abutting on the claw portion, and the claw portion includes a claw tip portion protruding from the wall portion,

the plunger includes a claw-flicking rib provided on a side surface of the distal end portion,

the claw-flicking rib is located on a rear side of the claw tip portion in a state where the plunger does not abut on the gasket pressing assembly in a pressable manner, and the claw-flicking rib is located on a lateral side of the claw tip portion in a state where the plunger abuts on the gasket pressing assembly in a pressable manner, and

as the plunger is rotated, the plunger advances to move the gasket pressing assembly forward, the claw-flicking rib abuts on the claw tip portion and passes the claw portion while deforming the claw portion, and a sound is generated as the claw portion restored after the passage abuts on the wall portion.

Also disclosed is a liquid medicine administration tool including: the above-described gasket pressing tool for a syringe; and a prefilled syringe that includes a barrel, a liquid medicine charged in the barrel, and the gasket slidably accommodated in the barrel, wherein the prefilled syringe is mounted on the syringe-mounting portion of the gasket pressing tool for a syringe.

### BRIEF DESCRIPTION OF THE DRAWINGS

FIG. 1 is a front view of a liquid medicine ejection tool according to an embodiment.

FIG. 2 is a cross-sectional view taken along line A-A of FIG. 1.

FIG. 3 is a front view of the liquid medicine ejection tool according to the embodiment in a state where a gasket pressing assembly is mounted on a gasket.

FIG. 4 is a left side view of the liquid medicine ejection tool illustrated in FIG. 3.

FIG. 5 is an enlarged bottom view of the liquid medicine ejection tool illustrated in FIG. 3.

FIG. 6 is an enlarged cross-sectional view taken along line B-B of FIG. 3.

FIG. 7 is a cross-sectional view taken along line C-C of FIG. 3.

FIG. 8 is a cross-sectional view taken along line D-D of FIG. 4.

FIG. 9 is a perspective view of the liquid medicine ejection tool illustrated in FIG. 3.

FIG. 10 is an explanatory view for describing components used in the liquid medicine ejection tool illustrated in FIG. 3.

FIG. 11 is a front view of the gasket pressing assembly used in the liquid medicine ejection tool illustrated in FIG. 3.

FIG. 12 is a bottom view of the gasket pressing assembly illustrated in FIG. 11.

FIG. 13 is a cross-sectional view taken along line E-E of FIG. 11.

FIG. 14 is an enlarged view of the vicinity of the gasket of FIG. 7.

FIG. 15 is a front view of the gasket used in the liquid medicine ejection tool.

FIG. 16 is a bottom view of the gasket illustrated in FIG. 15.

FIG. 17 is a vertical cross-sectional view of the gasket illustrated in FIG. 15 (when the gasket is not inserted into a barrel and the gasket pressing member is not mounted).

FIG. 18 is an explanatory view for describing a gasket shape when the gasket used in the liquid medicine ejection tool is inserted into the barrel and the gasket pressing member is mounted.

FIG. 19 is a front view of a gasket pressing member used in the gasket pressing assembly illustrated in FIG. 11.

FIG. 20 is a cross-sectional view taken along line F-F of FIG. 19.

FIG. 21 is a bottom view of the gasket pressing member illustrated in FIG. 19.

FIG. 22 is a perspective view of the gasket pressing member illustrated in FIG. 19 as seen obliquely from below.

FIG. 23 is a front view of a sound generating member used in the gasket pressing assembly illustrated in FIG. 11.

FIG. 24 is a bottom view of the sound generating member illustrated in FIG. 23.

FIG. 25 is a cross-sectional view taken along line G-G of FIG. 23.

FIG. 26 is a view obtained by illustrating a left side view of a syringe-mounting member used in the liquid medicine ejection tool illustrated in FIG. 3 sideways.

FIG. 27 is a bottom view of the syringe-mounting member in the state illustrated in FIG. 26.

FIG. 28 is a cross-sectional view taken along line H-H of FIG. 26.

FIG. 29 is a cross-sectional view taken along line I-I of FIG. 27.

FIG. 30 is a reference view of the syringe-mounting member of FIG. 26.

FIG. 31 is an explanatory view of an end surface along line Y-Y of FIG. 30.

FIG. 32 is an explanatory view of an end surface along line X-X of FIG. 30.

FIG. 33 is an explanatory view of an end surface along line Z-Z of FIG. 30.

FIG. 34 is a perspective view of the gasket pressing member in the state illustrated in FIG. 26 as seen obliquely from above.

FIG. 35 is a perspective view of the gasket pressing member in the state illustrated in FIG. 26 as seen obliquely from below.

FIG. 36 is an enlarged front view of a plunger used in the liquid medicine ejection tool illustrated in FIG. 3.

FIG. 37 is a left side view of the plunger illustrated in FIG. 36.

FIG. 38 is an enlarged perspective view of a distal end portion of the plunger illustrated in FIG. 36.

FIG. 39 is an explanatory cross-sectional view for describing a sound generating mechanism in the liquid medicine ejection tool.

FIG. 40 is an explanatory cross-sectional view for describing the sound generating mechanism in the liquid medicine ejection tool.

FIG. 41 is an explanatory cross-sectional perspective view for describing the sound generating mechanism in the liquid medicine ejection tool.

FIG. 42 is an explanatory cross-sectional view for describing the sound generating mechanism in the liquid medicine ejection tool.

FIG. 43 is an explanatory cross-sectional view for describing the sound generating mechanism in the liquid medicine ejection tool.

FIG. 44 is an explanatory cross-sectional perspective view for describing the sound generating mechanism.

FIG. 45 is a front view of a liquid medicine ejection tool according to another embodiment in a state where a gasket pressing assembly is mounted on a gasket.

FIG. 46 is a vertical cross-sectional view of the liquid medicine ejection tool illustrated in FIG. 45.

FIG. 47 is a perspective view of the liquid medicine ejection tool illustrated in FIG. 45.

FIG. 48 is a view obtained by illustrating a left side view of a syringe-mounting member used in the liquid medicine ejection tool illustrated in FIG. 45 sideways.

FIG. 49 is a bottom view of the syringe-mounting member in the state illustrated in FIG. 48.

FIG. 50 is a cross-sectional view taken along line J-J of FIG. 48.

FIG. 51 is a cross-sectional view taken along line K-K of FIG. 49.

FIG. 52 is a left side view of the syringe-mounting member in the state illustrated in FIG. 48.

FIG. 53 is a perspective view of the syringe-mounting member in the state illustrated in FIG. 48 as seen from the distal side.

FIG. 54 is a perspective view of the syringe-mounting member in the state illustrated in FIG. 48 as viewed from the proximal side.

FIG. 55 is an enlarged vertical cross-sectional view of a gasket pressing assembly used in the liquid medicine ejection tool illustrated in FIG. 45.

FIG. 56 is an explanatory view for describing components of the gasket pressing assembly used in the liquid medicine ejection tool illustrated in FIG. 45.

FIG. 57 is an enlarged bottom view of a gasket pressing member used in the liquid medicine ejection tool illustrated in FIG. 45.

FIG. 58 is an enlarged plan view of a joint member used in the liquid medicine ejection tool illustrated in FIG. 45.

FIG. 59 is an enlarged front view of a plunger used in the liquid medicine ejection tool illustrated in FIG. 45.

FIG. 60 is an enlarged view of a distal end portion of the plunger illustrated in FIG. 59.

FIG. 61 is a vertical cross-sectional view of a liquid medicine administration tool according to still another embodiment.

FIG. 62 is an explanatory view for describing a treatment method using the liquid medicine administration tool.

FIG. 63 is an explanatory view for describing the treatment method using the liquid medicine administration tool.

FIG. 64 is an explanatory view for describing the treatment method using the liquid medicine administration tool.

FIG. 65 is an explanatory view for describing the treatment method using the liquid medicine administration tool.

FIG. 66 is an explanatory view for describing the treatment method using the liquid medicine administration tool.

### DETAILED DESCRIPTION

Set forth below with reference to the accompanying drawings is a detailed description of embodiments of a gasket pressing tool for a syringe, a liquid medicine ejection tool, and a liquid medicine administration tool representing examples of the inventive gasket pressing tool for a syringe, liquid medicine ejection tool, and liquid medicine administration tool.

As a first configuration, a liquid medicine ejection tool 1 includes: a syringe 2 that includes a barrel 20 having a flange 23 at its proximal end portion and a gasket 7 slidable in the barrel 20 and elastically deformable; and a gasket pressing tool (gasket pressing tool for a syringe) 3 used by being mounted on the syringe 2.

The gasket 7 includes a tubular portion 72, a distal closing portion 71 closing a distal end of the tubular portion 72, a lumen portion 70 defined by the tubular portion 72 and the distal closing portion 71, and an annular rib (in the embodiment, three annular ribs 73, 74 and 75) provided on an outer surface of the tubular portion 72.

The gasket pressing tool 3 includes: a gasket pressing member 8 having a gasket-mounting portion (gasket entering portion) 80, which can enter the lumen portion 70 of the gasket 7 and made of a material harder than the gasket 7, and a gasket proximal surface pressing portion (flange portion) 81 provided at its proximal end portion of the gasket-mounting portion 80; a syringe-mounting member 4 that movably accommodates the gasket pressing member 8 and has a proximal barrel-mounting portion (barrel mounting slot) 43 which can be mounted to a proximal end portion of the barrel 20 having the flange 23; and a plunger 5 that has a distal end portion entering the syringe-mounting member 4 and can press the gasket pressing member 8 in a distal direction.

The gasket 7 has an original lumen portion axial length S when the gasket 7 is not inserted into the barrel 20 and the gasket pressing member 8 is not mounted, the gasket-mounting portion 80 of the gasket pressing member 8 has an axial length T, and the axial length T is longer than the original lumen portion axial length S.

When the gasket 7 is inserted into the barrel 20 and the gasket pressing member 8 is mounted, a mounting portion distal surface 80a of the gasket-mounting portion 80 of the gasket pressing member 8 abuts on a lumen distal surface 76 of the lumen portion 70 of the gasket 7, and the gasket proximal surface pressing portion 81 of the gasket pressing member 8 becomes close to or abuts on a proximal surface of the gasket 7.

In addition, the liquid medicine administration tool includes: the liquid medicine ejection tool 1 described above; a liquid medicine 11 charged in the syringe 2; and a sealing member 21 sealing the distal end portion of the barrel 20.

In addition, in the liquid medicine ejection tool 1 of the present embodiment, when the gasket 7 is inserted into the barrel 20 and the gasket pressing member 8 is mounted, an inner surface 77 of the lumen portion 70 of the gasket 7 is in close contact with an outer surface of the gasket-mounting portion 80 of the gasket pressing member 8, the gasket 7 is turned into a state of being stretched in the axial direction by the gasket pressing member 8, and the gasket proximal surface pressing portion 81 of the gasket pressing member 8 is turned into a state of being capable of pressing a proximal surface 79 of the gasket 7.

As a second configuration, a gasket pressing tool 3 for a syringe is used by being mounted to a syringe 2 that includes: a barrel 20 having an annular flange 23 at its proximal end portion; and a gasket 7 slidably accommodated in the barrel 20.

The gasket pressing tool 3 for a syringe includes: a gasket pressing member 8 configured to press the gasket 7; a tubular syringe-mounting member 4 that movably accommodates the gasket pressing member 8 and can be mounted on the proximal end portion of the barrel 20; and a plunger 5 that has a distal end portion entering the syringe-mounting member 4 and can press a gasket pressing assembly (gasket-mounting member) 6 having the gasket pressing member 8 by rotation.

The plunger 5 includes: a distal end portion that can press the gasket pressing member 8; a shaft portion 51 that extends from its distal end portion in a proximal direction and has a shaft-side screwing portion 55 on its outer peripheral surface; and a handle 52 configured to rotate the plunger provided at the proximal end portion of the shaft portion 51.

The syringe-mounting member 4 includes: a body portion (barrel-mounting portion) 40 that can accommodate the flange 23 of the barrel 20; a tubular accommodation portion 42 that extends from a proximal end of the body portion 40 in the proximal direction of the gasket pressing tool 3 and can accommodate the gasket pressing member; and a tubular insertion portion 110 (lumen 45) which extends from a proximal end of the tubular accommodation portion 42 (accommodation portion 47) in the proximal direction of the gasket pressing tool 3 and into which the shaft portion 51 of the plunger 5 is inserted. The insertion portion 110 (lumen 45) of the syringe-mounting member 4 has an insertion-portion-side screwing portion 46, which is screwed with the shaft-side screwing portion of the shaft portion 51, on its inner peripheral surface.

The body portion 40 includes: a distal-side support portion 103 that can support a distal surface of the flange 23 (in other words, can abut on the distal surface of the flange 23); a proximal-side support portion 104 that can support a proximal surface of the flange 23 (in other words, can abut on the proximal surface of the flange 23); a connecting portion 105 that connects the distal-side support portion 103 and the proximal-side support portion 104; a flange accommodation portion 102 that is formed of the distal-side support portion 103, the proximal-side support portion 104, and the connecting portion 105 and can accommodate the flange 23; and a flange insertion opening 101 that is open to a lateral side of the body portion 40 and configured to insert the flange into the flange accommodation portion 102.

The distal-side support portion 103 has an axial thickness (N) of 5 to 25 mm along a central axis of the syringe-mounting member 4, and the proximal-side support portion 104 has an axial thickness (M) of 5 to 25 mm along the central axis of the syringe-mounting member 4. In addition, the connecting portion 105 has a radial width (Ta) of 1 to 5 mm along the radial direction of the syringe-mounting member 4.

In addition, a liquid medicine administration tool may include: a prefilled syringe which includes a barrel 20, a liquid medicine 11 charged in the barrel 20, a gasket 7 slidably accommodated in the barrel 20, and a sealing member (sealing cap) 21 sealing a distal end of the barrel 20; and a gasket pressing tool 3 on which the prefilled syringe is mounted.

The prefilled syringe does not necessarily include a plunger. The barrel 20 includes a nozzle 22 for discharging a liquid medicine and a collar portion 24 provided so as to cover the nozzle 22 at its distal end, and includes the flange 23 protruding outward at a rear end portion.

As a third configuration, a gasket pressing tool 3 for a syringe is used by being mounted to a syringe 2 that includes: a barrel 20; and a gasket 7 slidably accommodated in the barrel 20.

The gasket pressing tool 3 for a syringe includes: a gasket pressing assembly 6 including a gasket pressing member 8 and a sound generating member 9; a syringe-mounting member 4 which includes a proximal barrel-mounting portion 43 and movably accommodates the gasket pressing assembly 6; and a plunger 5 which enters the syringe-mounting member 4 on a distal side and can press (can press and advance) the gasket pressing assembly 6 by rotation (advancing along with rotation).

The sound generating member 9 includes claw portions 92 and 93 that are elastically deformable (specifically, elastically deformable in the circumferential direction with respect to an axis of the gasket pressing member 8). The gasket pressing member 8 includes wall portions 88 and 89 that abut on (specifically, abut on and press) the claw portions 92 and 93, respectively. The claw portions 92 and 93 include claw tip portions 92a and 93a protruding rearward from the wall portions 88 and 89, respectively. The plunger 5 includes claw-flicking ribs 54 provided on a side surface of the distal end portion. In a state where the plunger 5 (specifically, the distal end portion) does not abut on the gasket pressing assembly 6 in a pressable manner (non-pressing state), the claw-flicking rib 54 is located on the rear side of each of the claw tip portions 92a and 93a. In a state where the plunger 5 (specifically, the distal end portion) abuts on the gasket pressing assembly 6 in a pressable state (pressable state), the claw-flicking rib 54 is located on the lateral side of each of the claw tip portions 92a and 93a. As the plunger 5 is rotated, the plunger 5 advances to move the gasket pressing assembly 6 forward, the claw-flicking ribs 54 abut on the claw tip portions 92a and 93a and pass the claw portions 92 and 93 while deforming the claw portions 92 and 93. As the claw portions 92 and 93 restored after the passage abut on the wall portions 88 and 89, a sound is generated.

In addition, a liquid medicine administration tool may include: a prefilled syringe which includes a barrel 20, a liquid medicine 11 charged in the barrel 20, a gasket 7 slidably accommodated in the barrel 20, and a sealing member (sealing cap) 21 sealing a distal end of the barrel 20; and a gasket pressing tool 3 on which the prefilled syringe is mounted.

The prefilled syringe does not necessarily include a plunger. The barrel 20 includes a nozzle 22 for discharging a liquid medicine and a collar portion 24 provided so as to cover the nozzle 22 at its distal end, and includes the flange 23 protruding outward at a rear end portion.

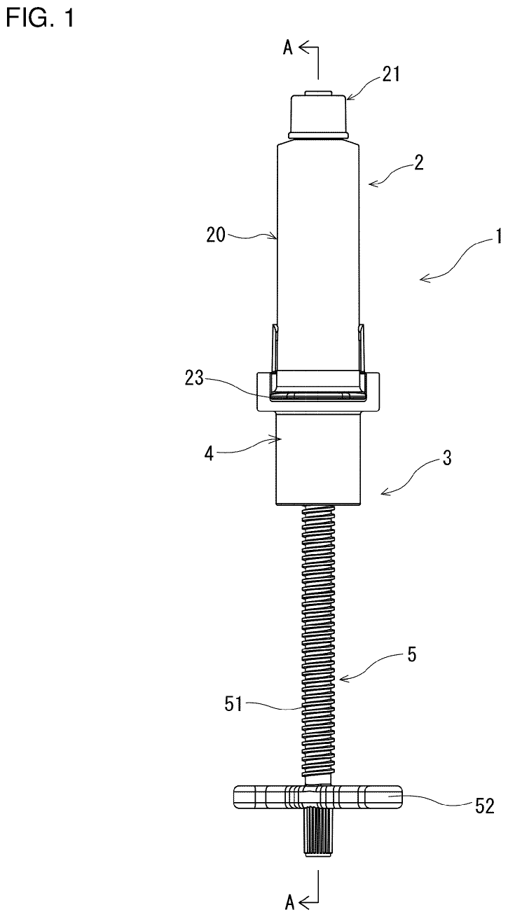

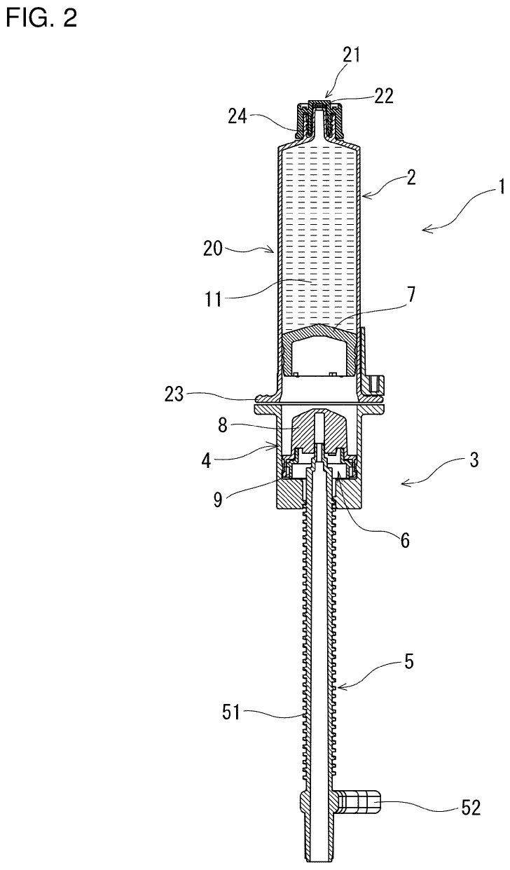

The liquid medicine administration tool (liquid medicine ejection tool 1) illustrated in FIGS. 1 and 2 includes: the prefilled syringe that includes the syringe 2, the liquid medicine 11 charged in the syringe 2, and the sealing member (sealing cap) 21 sealing the distal end portion of the barrel 20; and the gasket pressing tool 3 on which the prefilled syringe is mounted. The prefilled syringe does not include a plunger. The barrel 20 includes a nozzle 22 for discharging a liquid medicine and a collar portion 24 provided so as to cover the nozzle 22 at its distal end, and includes the flange 23 protruding outward at a proximal end portion.

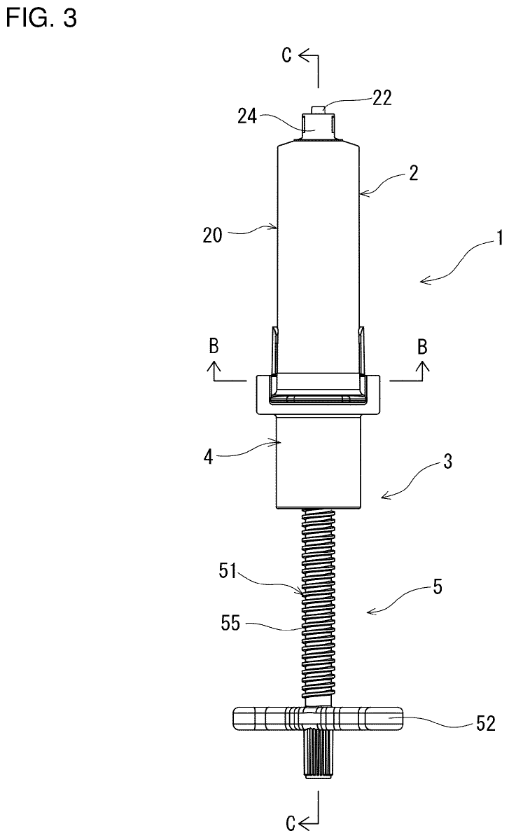

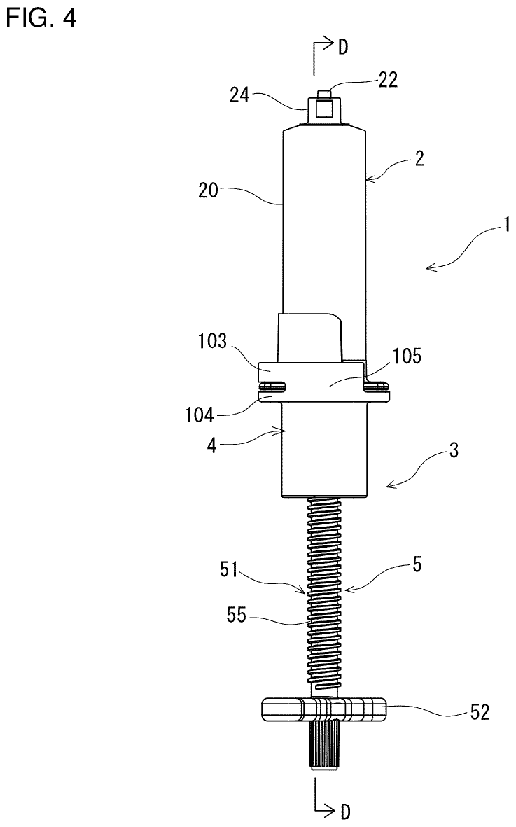

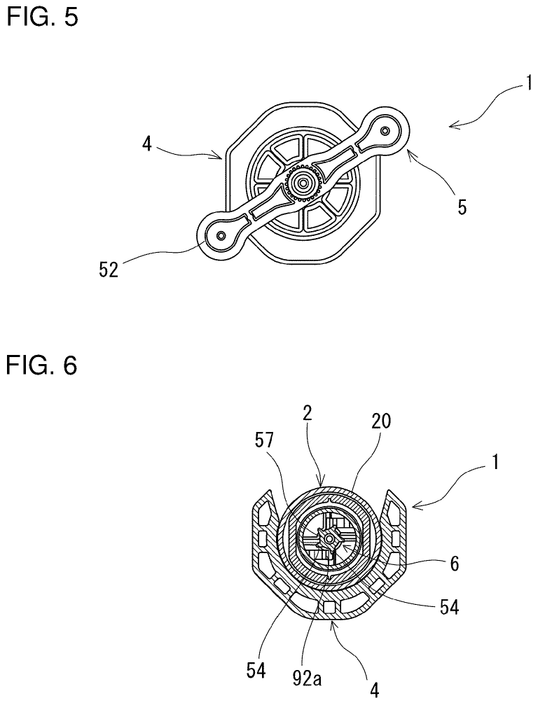

In the liquid medicine ejection tool 1 illustrated in FIGS. 1 and 2, the gasket pressing assembly 6 including the gasket pressing member 8 is located on the rear side of the gasket 7 and is not mounted on the gasket 7. As the plunger 5 is rotated to move forward during use, the gasket pressing assembly 6 is mounted to the gasket 7 as illustrated in FIGS. 3 and 4 to 6. The gasket pressing assembly 6 can also be referred to as a gasket-mounting member. Note that the liquid medicine and the sealing cap are not illustrated in the syringe 2 in FIG. 3 and the subsequent drawings.

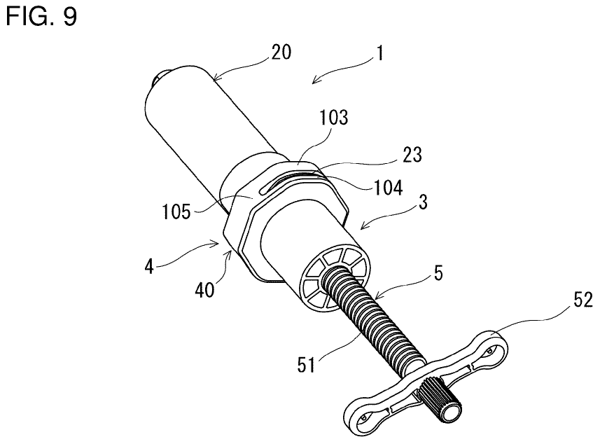

As illustrated in FIGS. 1 to 9, the liquid medicine ejection tool 1 includes the gasket pressing tool 3. As described above, the gasket pressing assembly 6 is mounted to the gasket 7 in the structure illustrated in FIGS. 3 and 4 to 6.

The gasket pressing tool 3 includes: the gasket pressing assembly 6; the plunger 5 configured to press the gasket pressing assembly 6 from the rear side; and the syringe-mounting member 4 configured to mount the gasket pressing assembly 6 and the plunger 5 onto the syringe 2.

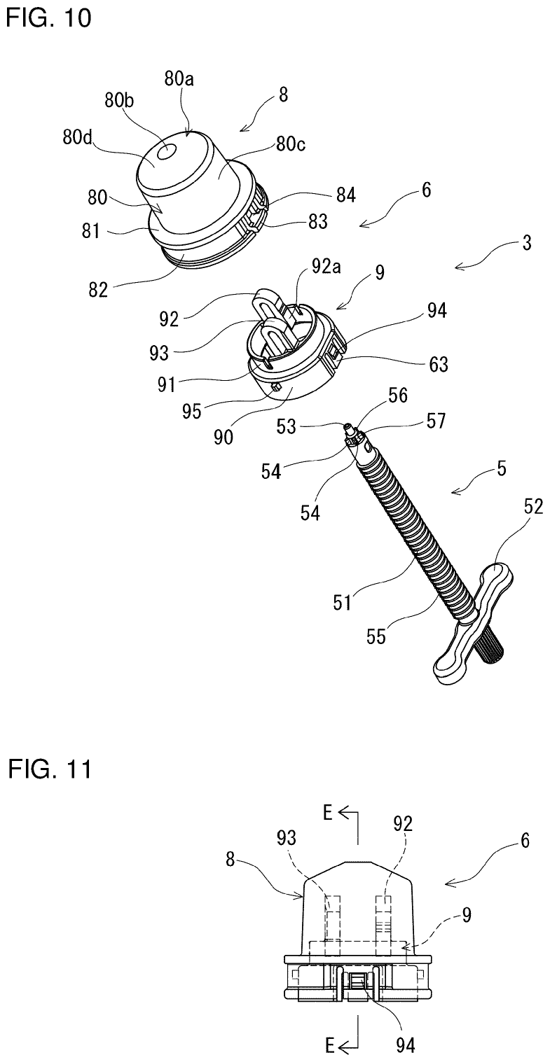

As illustrated in FIGS. 10 to 13, the gasket pressing assembly 6 includes: the gasket pressing member 8 and the sound generating member 9 mounted on the gasket pressing member 8.

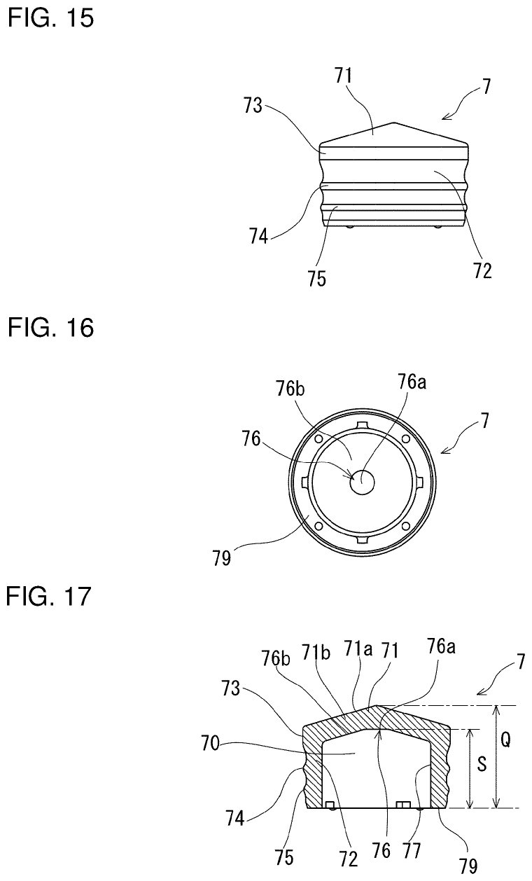

As illustrated in FIGS. 15 to 18, the gasket 7 includes: the lumen portion 70; the tubular portion 72; the distal closing portion 71 that closes the distal end of the tubular portion 72; and annular ribs 73, 74, and 75 provided on an outer surface of the tubular portion 72.

The gasket 7 in FIGS. 15 to 17 has a form in the state of not being inserted into the barrel 20 and having no gasket-mounting member 8 mounted thereon. As illustrated in FIG. 17, the gasket 7 has the original lumen portion axial length S when the gasket is not inserted into the syringe and the gasket-mounting member is not mounted. In addition, the lumen portion 70 of the tubular portion 72 extends in the distal direction with the substantially same inner diameter. In addition, the inner surface of the lumen portion 70 includes: the lumen distal surface 76; and a proximal-side cylindrical surface (inner surface) 77 extending rearward from a proximal end of the lumen distal surface 76 with substantially the same diameter. Further, the lumen distal surface 76 has: a flat central flat surface 76a provided at the center of the lumen distal surface; and a lumen portion tapered surface 76b extending rearward from a proximal end of the central flat surface 76a.

Specifically, as illustrated in FIG. 17, the gasket 7 has: the lumen distal surface 76 of the lumen portion 70 of the gasket 7; the flat central flat surface 76a provided at the center of the lumen distal surface; and the central flat surface 76a has a lumen portion tapered surface 76b extending rearward and radially outward from an outer edge of the central flat surface 76a. In addition, the distal closing portion 71 of the gasket 7 has: the tapered gasket distal surface 71a whose diameter is reduced toward a distal end; and a uniform-thickness portion 71b which is formed between the gasket distal surface and the lumen portion tapered surface and has a substantially uniform thickness.

As illustrated in FIGS. 19 to 22, the gasket pressing member 8 includes: the gasket-mounting portion 80 that enters the gasket 7 and comes into contact with an inner surface of the gasket; and the gasket proximal surface pressing portion 81 formed at a rear portion of the gasket-mounting portion 80. The gasket-mounting portion 80 of the gasket pressing member 8 has the axial length T, and the axial length T is longer than the original lumen portion axial length S of the gasket 7. Further, the axial length T is preferably 1.05 to 1.20 times, and particularly preferably 1.08 to 1.15 times of the original lumen portion axial length S.

Further, as illustrated in FIG. 14, when the gasket 7 is inserted into the barrel 20 and the gasket pressing member 8 is mounted, the inner surface 77 of the lumen portion 70 of the gasket 7 is in close contact with the outer surface of the gasket-mounting portion 80 of the gasket pressing member 8, and the gasket 7 is turned into a state of being stretched in the axial direction by the gasket pressing member 8.

In addition, the gasket-mounting portion 80 includes: the distal end portion having the mounting portion distal surface 80a; and a proximal-side tapered portion 80c which extends from the distal end portion of the gasket-mounting portion 80 (the mounting portion distal surface 80a) to the gasket proximal surface pressing portion 81 and is enlarged in diameter toward the rear side to have a tapered shape. The mounting portion distal surface 80a includes a flat distal surface 80b and a distal-side tapered surface 80d extending rearward from a proximal end of the flat distal surface 80b.

When the gasket 7 is inserted into the barrel 20 and the gasket pressing member 8 is mounted, the mounting portion distal surface 80a of the gasket-mounting portion 80 of the gasket pressing member 8 abuts on the lumen distal surface 76 of the lumen portion 70 of the gasket 7.

In addition, the proximal-side tapered portion 80c of the gasket-mounting portion 80 becomes close to or abuts on an inner side surface of the lumen portion 70 of the gasket 7. In the present embodiment, the proximal-side tapered portion 80c abuts on the proximal-side cylindrical surface (inner surface) 77 of the lumen portion 70 of the gasket 7. A taper angle of the proximal-side tapered portion of the gasket-mounting portion 80 is preferably 1 to 8 degrees, and particularly preferably 3 to 6 degrees.

As illustrated in FIG. 14, when being mounted on the gasket 7, the gasket pressing member 8 is pushed into the gasket 7 to deform and stretch the gasket 7 in a state where the central flat surface 76a of the lumen distal surface 76 of the lumen portion 70 of the gasket 7 abuts on the flat distal surface 80b of the mounting portion flat surface 80a of the gasket-mounting portion 80 and the lumen portion tapered surface 76b of the lumen portion 70 abuts on the distal-side tapered surface 80d of the gasket-mounting portion 80. In addition, in the present embodiment, the entire outer surface of the gasket-mounting portion 80 has a shape that substantially coincides with the entire inner surface of the lumen portion 70 of the gasket 7 when being inserted into the barrel 20.

Specifically, the mounting portion distal surface 80a of the gasket-mounting portion 80 has: the flat distal surface 80b, which is flat, at the center of the mounting portion distal surface; and the tapered outer edge surface (distal-side tapered surface) 80d extending rearward and radially outward from the outer edge of the flat distal surface 80b. When the gasket 7 is inserted into the barrel 20 and the gasket pressing member 8 is mounted, the flat distal surface 80b of the gasket-mounting portion 80 abuts on the central flat surface 76a of the lumen portion 70 of the gasket 7, and the tapered outer edge surface 80d of the gasket-mounting portion 80 becomes close to or abuts on the lumen portion tapered surface 76b of the lumen portion 70 of the gasket.

Due to the deformation including stretching of the gasket 7, a front surface of the inner surface 77 of the lumen portion 70 of the gasket 7 is in close contact with the outer surface of the gasket-mounting portion 80 of the gasket pressing member 8. In this state, the lumen portion of the gasket 7 is not deformable practically. Even if the gasket 7 in the state where the lumen portion is not deformable is elastically deformed, the volume does not change. Accordingly, even if a needle tip for administration is removed after the end of administration, the volume of the gasket does not change, so that the outflow of the liquid medicine caused by the shape change of the gasket does not occurs.

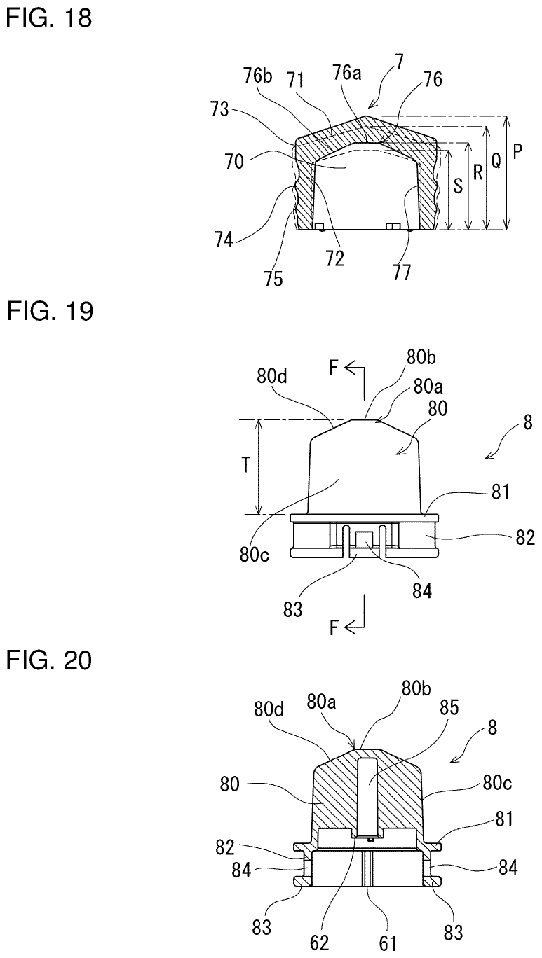

As illustrated in FIG. 17, the gasket 7 has an original axial length Q and the original lumen portion axial length S in the state of not being inserted into the barrel 20 (syringe 2) and having no gasket-mounting member 8 mounted thereon. In addition, as illustrated in FIG. 18, the gasket 7 has a deformed axial length P and a deformed lumen portion axial length R in the state of being inserted into the syringe and having the gasket-mounting member mounted thereon. As illustrated in FIG. 18, the deformed axial length P is longer than the original axial length Q, and the deformed lumen portion axial length R is longer than the original lumen portion axial length S. The deformed axial length P is preferably longer than the original axial length Q by 0.5 to 3 mm, and particularly preferably by 1 to 2 mm. In addition, the deformed lumen portion axial length R is preferably longer than the original lumen portion axial length S by 0.5 to 3 mm, and particularly preferably by 1 to 2 mm.

As illustrated in FIG. 19, the gasket pressing member 8 has the axial length T of the gasket-mounting portion. The axial length T of the gasket pressing member 8 is preferably 1.05 to 1.20 times, and particularly preferably 1.08 to 1.15 times of the original lumen portion axial length S of the gasket 7. In addition, the taper angle of the proximal-side tapered portion 80c of the gasket-mounting portion 80 of the gasket pressing member 8 is preferably 1 to 8 degrees, and particularly preferably 3 to 6 degrees.

In addition, the gasket pressing member 8 includes a cylindrical portion 82 extending rearward from the gasket proximal surface pressing portion 81. Further, the gasket-mounting portion 80 includes a distal closing portion and a tapered diameter-reducing portion extending from the gasket proximal surface pressing portion 81 to a proximal end of the distal closing portion. The gasket pressing member 8 includes two deformable portions 83 provided in the cylindrical portion 82 and facing each other, and engagement window portions 84 provided in the respective deformable portions. The deformable portion 83 and the engagement window portion 84 form an engagement portion with respect to the sound generating member 9.

Further, the gasket pressing member 8 includes: a sound generating member accommodation portion, the axially extending wall portions 88 and 89, a plunger abutment portion 62, and a columnar recess 85 therein as illustrated in FIGS. 10 to 14 and 19 to 22. The plunger abutment portion 62 of the gasket pressing member 8 can abut on and be separated from the distal end portion of the plunger 5.

Further, in the state where the gasket 7 is inserted into the barrel 20 and the gasket pressing member 8 is mounted, when the plunger 5 is advanced in the distal direction, the distal end portion of the plunger 5 abuts on the plunger abutment portion 62 of the gasket pressing member 8. When the plunger 5 is pulled in the proximal direction, the mounting state of the gasket pressing member 8 on the gasket 7 is maintained, and the plunger abutment portion 62 is separated from the distal end portion of the plunger. For this reason, the operation of the plunger 5 does not cause the gasket 7 and the gasket pressing member 8 to retract.

In addition, in the state where the gasket 7 is inserted into the barrel 20 and the gasket-mounting portion 80 is inserted into the lumen portion 70 of the gasket 7, the mounting portion distal surface of the gasket-mounting portion 80 is kept in the state of abutting on the lumen distal surface of the lumen portion 70 of the gasket 7 even if the plunger 5 is pulled in the proximal direction. For this reason, even if the pressure on the gasket-mounting portion 80 is released, the liquid medicine (highly viscous substance), which has flowed out once, is prevented from being drawn back into the barrel 20.

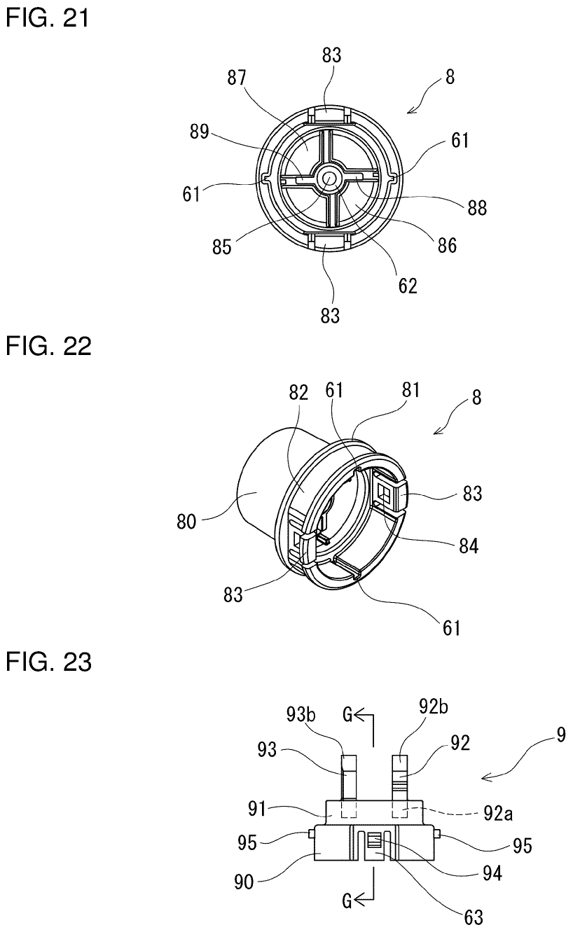

As illustrated in FIG. 21, the gasket pressing member 8 is provided with the cylindrical portion having the columnar recess 85 at the center of the inside, and provided with four partition portions extending laterally from the cylindrical portion. The four partition portions form four axially extending spaces inside the gasket pressing member 8. The wall portions 88 and 89 are formed by two partition portions facing each other with a slight shift from a central axis of the cylindrical portion formed inside the gasket pressing member. In addition, the plunger abutment portion 62 is formed by a proximal surface of the cylindrical portion having the columnar recess 85. The columnar recess 85 is a concave portion having a cylindrical shape.

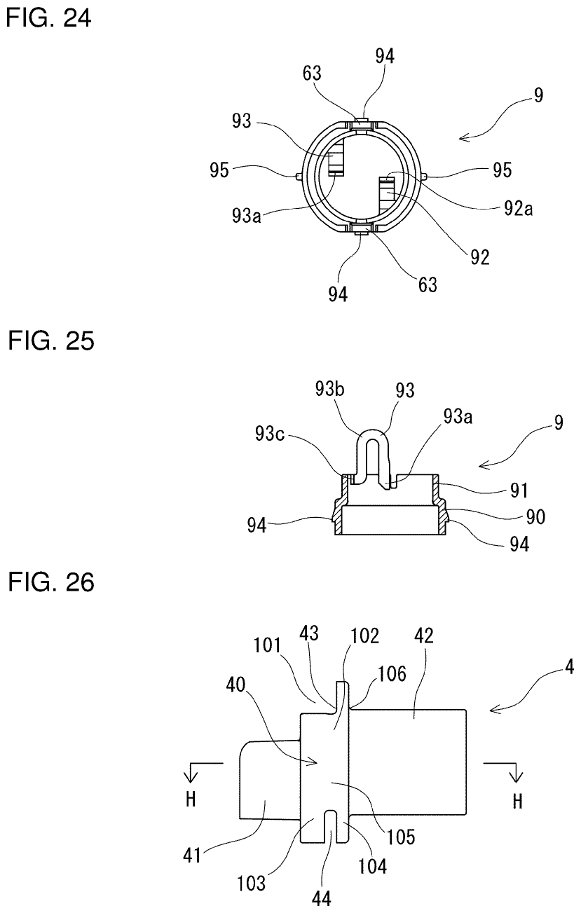

The sound generating member 9 includes: a tubular body portion 91, the elastically deformable claw portions 92 and 93 protruding in the distal direction from an inner surface of the tubular body portion, and an enlarged-diameter tubular portion 90 extending downward from the tubular body portion 91 as illustrated in FIGS. 10 to 13 and 23 to 25. In the present embodiment, the two claw portions 92 and 93 are provided, and the claw tip portions 92a and 93a substantially face each other.

The claw portions 92 and 93 include: base portions 92c and 93c connected to an inner surface of a distal end portion of the tubular body portion 91; and curved portions 92b and 93b protruding forward from the base portions 92c and 93c; and the claw tip portions 92a and 93a which are free ends extending from the curved portions 92b and 93b and facing the proximal direction (rear end direction). The curved portions 92b and 93b are continuous with the base portions 92c and 93c on one end side and are continuous with the claw tip portions 92a and 93a on the other end side. Further, distal end portions of the claw portions 92 and 93 are configured by the curved portions 92b and 93b. The claw tip portions 92a and 93a and the base portions 92c and 93c substantially face each other.

Further, the claw tip portions 92a and 93a are located inside the tubular body portion 91 in the present embodiment. The claw portions 92 and 93 can be elastically deformed, specifically, curved in a direction in which the claw tip portions 92a and 93a approach the base portions 92c and 93c. In addition, ends of the claw tip portions 92a and 93a are located closer to the proximal side than the base portions 92c and 93c.

In addition, the enlarged-diameter tubular portion 90 is provided with two deformable portions 63 facing each other and engagement ribs 94 provided on the respective deformable portions 63. The sound generating member 9 is mounted on the gasket pressing member 8 as the engagement ribs 94 of the sound generating member 9 engage with the engagement window portions 84 of the gasket pressing member 8. In addition, the enlarged-diameter cylindrical portion 90 is provided with side ribs 95, and the side ribs 95 enter two recesses 61 which are provided on the inner surface of the cylindrical portion 82 of the gasket pressing member 8 and face each other. For this reason, the rotation of the sound generating member 9 inside the gasket pressing member 8 is regulated.

Then, the distal end portion of the claw tip portion 93a of the sound generating member 9 is located on the rear side (in other words, protrudes) from a lower end of the wall portion 89 of the gasket pressing member 8 in the gasket pressing assembly 6 as illustrated in FIG. 13. Similarly, the distal end portion of the claw tip portion 92a of the sound generating member 9 is also located on the rear side (in other words, protrudes) from a lower end of the wall portion 88 of the gasket pressing member 8.

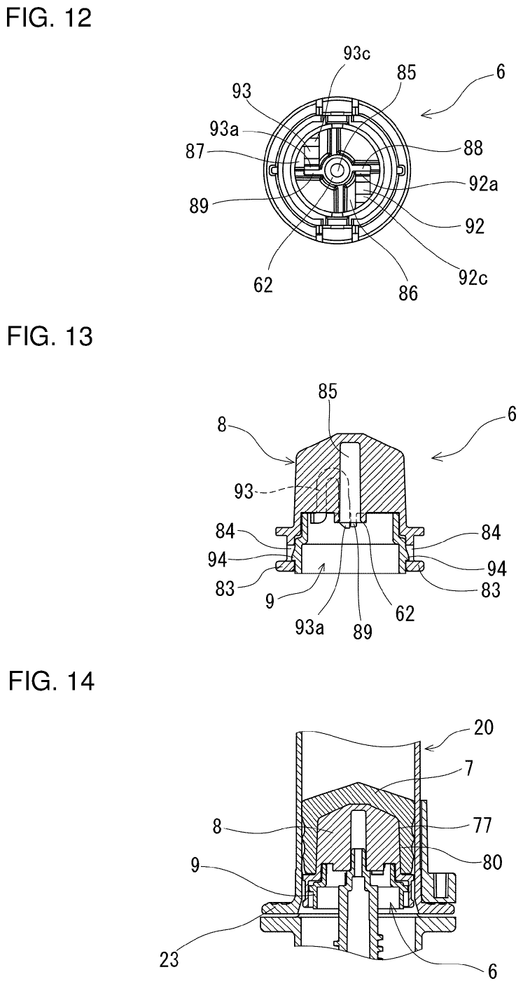

Further, tip-side side portions of the claw portions 92 and 93 of the sound generating member 9 abut on the wall portions 88 and 89 of the gasket pressing member 8, and are pressed by the wall portions 88 and 89 to be slightly curved (elastically deformed) toward the base portions 92c and 93c as illustrated in FIG. 12. In addition, the claw portions 92 and 93 are accommodated in space portions 86 and 87 divided by the partition portions inside the gasket pressing member 8.

In addition, the claw tip portions 92a and 93a are tapered in the present embodiment as illustrated in FIGS. 25, 40 and 41. In addition, abutment surfaces of the wall portions 88 and 89 and the claw portions 92 and 93 are formed as flat surfaces to generate a favorable sound. Specifically, surfaces of the claw tip portions 92a and 93a that respectively face the wall portions 88 and 89 (the surfaces that abut on the claw-flicking ribs 54 during rotation of the plunger 5 in a screw advancing direction to be described later) are flat surfaces. In addition, the flat surfaces of the claw tip portions 92a and 93a that abut on the claw-flicking ribs 54 are substantially parallel to a central axis of the sound generating member 9.

In addition, surfaces of the claw tip portions 92a and 93a close to the base portions 92c and 93c (the surfaces with which the claw-flicking ribs 54 are likely to come into contact during rotation of the plunger 6 in a screw retracting direction of to be described later) are formed as inclined surfaces that move away from the base portions 92c and 93c toward distal ends. In addition, the claw portions 92 and 93 extend upward from the base portions 92c and 93c and are curved in a U shape at the curved portions 92b and 93b, and the claw tip portions 93a and 93a are the free ends facing the proximal direction as illustrated in FIGS. 10 and 25. Further, portions, each close to the curved portion by a predetermined length, of the claw tip portions 92a and 93a are formed to be slightly thicker than the other portions. Each of the claw portions 92 and 93 has a rectangular cross section as a whole.

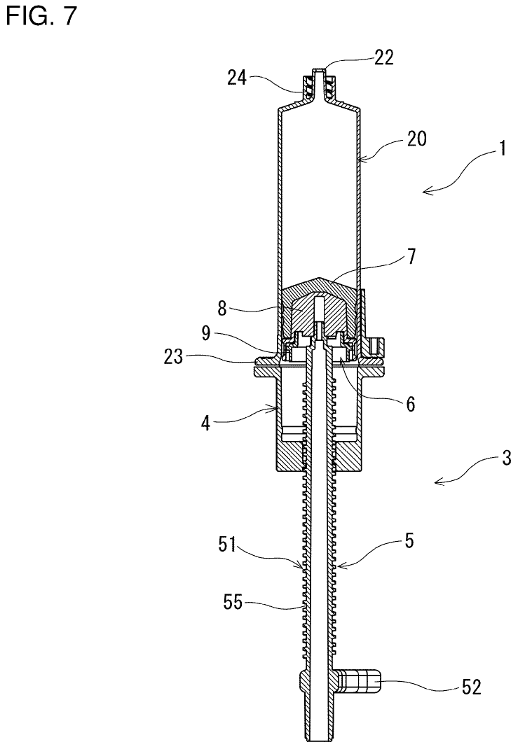

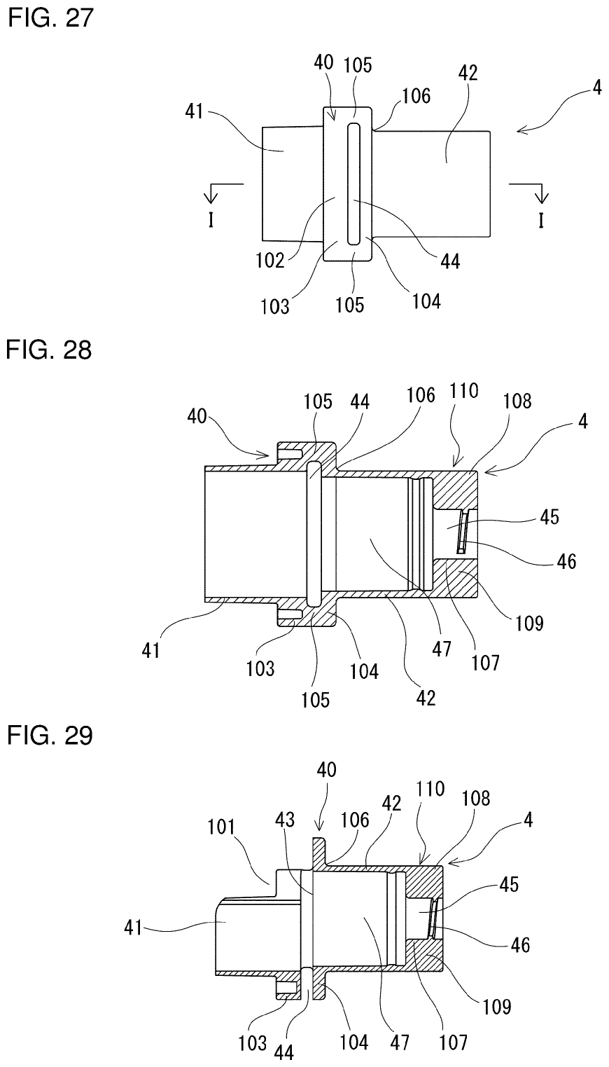

The syringe-mounting member 4 includes the barrel-mounting portion 43 as illustrated in FIGS. 1 to 9 and 26 to 35, and movably accommodates the gasket pressing assembly 6, particularly as illustrated in FIGS. 2 and 7.

The syringe-mounting member 4 includes the distal-side support portion 103, the proximal-side support portion 104, the connecting portion 105, the flange accommodation portion 102, and the flange insertion opening 101 as illustrated in FIGS. 26 to 29, 34, and 35. The distal-side support portion 103 can abut on a distal surface of the flange 23 of the barrel 20 accommodated in the flange accommodation portion 101, and supports the distal surface of the flange 23.

In the syringe-mounting member 4 of the present embodiment, the distal-side support portion 103 has a predetermined thickness (axial thickness: N) extending along the central axis of the syringe-mounting member 4 as illustrated in FIGS. 28 to 30, and the thickness (N) is preferably 5 to 25 mm, and particularly preferably 10 to 15 mm.

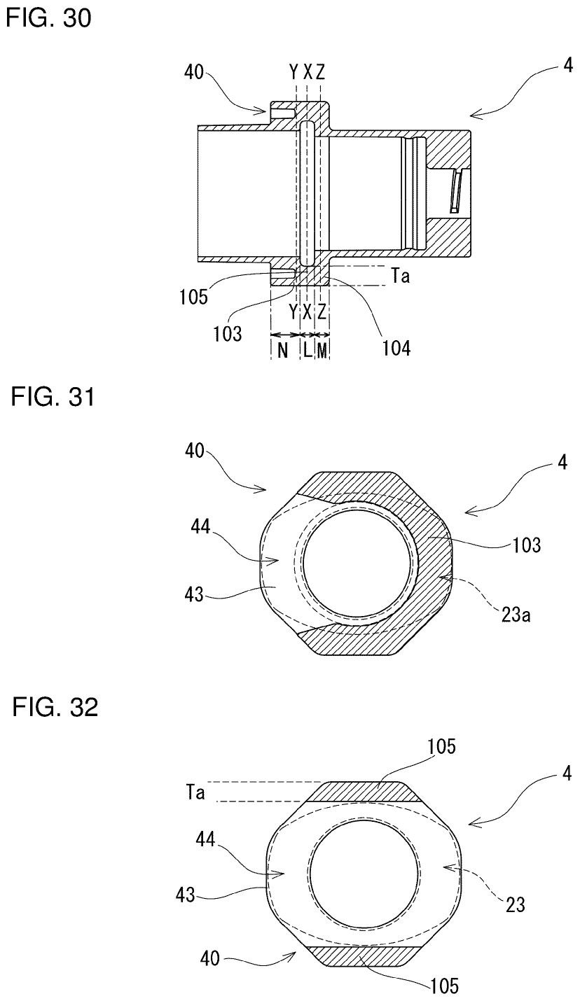

In addition, the distal-side support portion 103 has a shape and an area as illustrated in FIG. 31, which is a reference view illustrating an end surface along line Y-Y of FIG. 30, in the present embodiment. For this reason, the distal-side support portion 103 can abut on the flange 23 with a certain area as illustrated in FIG. 31. For this reason, the distal-side support portion 103 can sufficiently support the distal surface of the flange. The distal-side support portion 103 is preferably capable of supporting 55 to 80% of a distal surface 23a of the flange 23 (in other words, capable of abutting on 55 to 80% of the distal surface 23a), and particularly preferably capable of supporting 70 to 80% (capable of abutting on 70 to 80%) of the distal surface 23a.

In addition, in the syringe-mounting member 4 of the present embodiment, the proximal-side support portion 104 has a predetermined thickness (axial thickness: M) extending along the central axis of the syringe-mounting member 4 as illustrated in FIGS. 28 to 30, and the thickness (M) is preferably 5 to 25 mm, and particularly preferably 10 to 15 mm.

In addition, the proximal-side support portion 104 has a shape and an area as illustrated in FIG. 33, which is a reference view illustrating an end surface along line Z-Z of FIG. 30, in the present embodiment. For this reason, the proximal-side support portion 104 can abut on almost the entire proximal surface 23b of the flange 23 as illustrated in FIG. 33. For this reason, the proximal-side support portion 104 can sufficiently support the proximal surface of the flange.

In addition, the distal-side support portion 103 and the proximal-side support portion 104 are connected by the connecting portion 105 in the syringe-mounting member 4 of the present embodiment. Further, the connecting portion 105 has a predetermined thickness (axial thickness: L) extending along the central axis of the syringe-mounting member 4 as illustrated in FIGS. 28 to 30, and the thickness (L) is preferably −1 to 1 mm with respect to the thickness of the flange 23 of the barrel 20, and particularly preferably −0.5 to 0.5 mm with respect to the thickness of the flange 23 of the barrel 20.

In addition, the connecting portion 105 has a shape and an area as illustrated in FIG. 32, which is a reference view illustrating an end surface along line X-X of FIG. 30, in the present embodiment, and there are two connecting portions 105 facing each other. In addition, the connecting portion 105 has the radial width Ta along the radial direction of the syringe-mounting member 4 as illustrated in FIGS. 30 and 32. The radial width (Ta) is preferably 1 to 5 mm, and particularly preferably 2 to 3 mm.

The syringe-mounting member 4 of the present embodiment includes the flange accommodation portion 102 formed (defined) by the distal-side support portion 103, the proximal-side support portion 104, and the connecting portion 105. Further, the flange accommodation portion 102 includes: the flange insertion opening 101 open to the lateral side of the body portion 40 and configured to insert the flange into the flange accommodation portion 102; and a slot opening (opening portion) 44 which faces the flange insertion opening 101 (open to the lateral side of the body portion 40) and into which a part of the flange accommodated in the flange accommodation portion can enter.

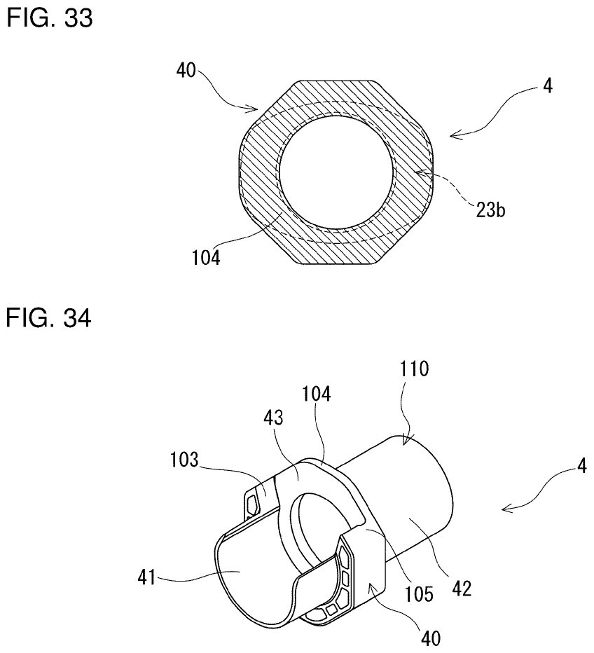

The syringe-mounting member 4 includes a proximal barrel-mounting portion 43 at a proximal end of the body portion 40. The proximal barrel-mounting portion 43 accommodates the flange 23 of the barrel 20 of the syringe 2. In the present embodiment, the proximal barrel-mounting portion 43 is formed using a slot (barrel mounting slot) into which the flange 23 of the syringe 2 can be inserted from one long diameter side (in other words, vertically inserted) as illustrated in FIG. 34. For this reason, in the state where the syringe 2 is mounted on the syringe-mounting member 4, one long-diameter portion of the flange 23 and two short-diameter portions facing each other of the syringe 2 are accommodated in the syringe-mounting member 4, and a long-diameter portion on the other end side is exposed. In addition, one end of the long-diameter portion of the flange accommodated in the syringe-mounting member 4 is exposed through the slot opening 44. In the present embodiment, the proximal barrel-mounting portion 43 is formed by a vertically elongated U-shaped slot.

In addition, in the syringe-mounting member 4 of the present embodiment, the body portion 40 has an outer peripheral surface (outer wall portion) extending along the central axis of the syringe-mounting member from a distal end of the distal-side support portion 103 to a proximal end of the proximal-side support portion 104 as illustrated in FIGS. 26 to 29, 34, and 35. Specifically, the outer peripheral surface (outer wall portion) has a semi-polygonal tubular shape in which a portion of the flange insertion opening 101 disappears.

In addition, in the present embodiment, the syringe-mounting member 4 includes a substantially semi-tubular portion 41 extending to the distal side from the body portion 40 having the proximal barrel-mounting portion (flange accommodation portion) 43 to hold the proximal end portion of the mounted syringe 2 as illustrated in FIGS. 26 to 35. In other words, the syringe-mounting member 4 includes the substantially semi-tubular portion (barrel support portion) 41 that extends from the flange accommodation portion 102 in the distal direction and supports the outer peripheral surface of the tubular portion of the barrel 20. The substantially semi-tubular portion 41 is formed by a substantially semi-tubular portion that extends to the distal side from the body portion 40 (flange accommodation portion 102).

Further, the syringe-mounting member 4 includes the tubular accommodation portion 42 capable of accommodating the gasket pressing member 8 (gasket pressing assembly 6) in the proximal direction from the proximal barrel-mounting portion 43 (body portion 40). The tubular accommodation portion 42 is a tubular portion that extends rearward from the proximal end of the body portion (barrel-mounting portion) 40. The tubular accommodation portion 42 includes the accommodation portion 47 for the gasket pressing assembly therein. The tubular accommodation portion 42 extends in the proximal direction with an outer diameter smaller than an outer diameter of the proximal-side support portion 104 of the body portion (barrel-mounting portion) 40.

Further, in the syringe-mounting member 4 of the present embodiment, the tubular accommodation portion 42 has a curved portion 106 that is enlarged in diameter toward the distal end and is connected to the proximal end of the proximal-side support portion of the body portion (barrel-mounting portion) 40 as illustrated in FIGS. 26 to 29. The curved portion 106 is formed in an annular shape. A radius of curvature of the curved portion 106 is preferably 0.3 to 3 mm, and particularly preferably 0.5 to 1.5 mm.

The accommodation portion 47 is formed inside the tubular accommodation portion 42. In other words, the syringe-mounting member 4 includes the tubular accommodation portion 47 capable of accommodating the gasket pressing member 8 (gasket pressing assembly 6) in the proximal direction from the proximal barrel-mounting portion 43 (body portion 40). Since such an accommodation portion 47 is provided, the gasket-mounting member 8 does not become an obstacle when the barrel 20 is mounted on the proximal barrel-mounting portion 43 of the syringe-mounting member 4.

Further, the syringe-mounting member 4 of the present embodiment includes the tubular insertion portion 110 which extends in the proximal direction of the gasket pressing tool 3 from the proximal end of the tubular accommodation portion 42 and into which the shaft portion 51 of the plunger 5 is inserted. The shaft portion 51 of the plunger 5 includes the shaft-side screwing portion 55, and the insertion-portion-side screwing portion 46 that is screwed with the shaft-side screwing portion 55 is provided on an inner peripheral surface of the insertion portion 110 (lumen 45). In other words, the insertion portion 110 (lumen 45) has the insertion-portion-side screwing portion 46 to be screwed with the shaft-side screwing portion 55 of the shaft portion 51 on its inner surface. In the present embodiment, the shaft-side screwing portion 55 is a spiral groove, and the insertion-portion-side screwing portion 46 is a spiral rib (spiral protrusion), specifically, a short spiral rib.

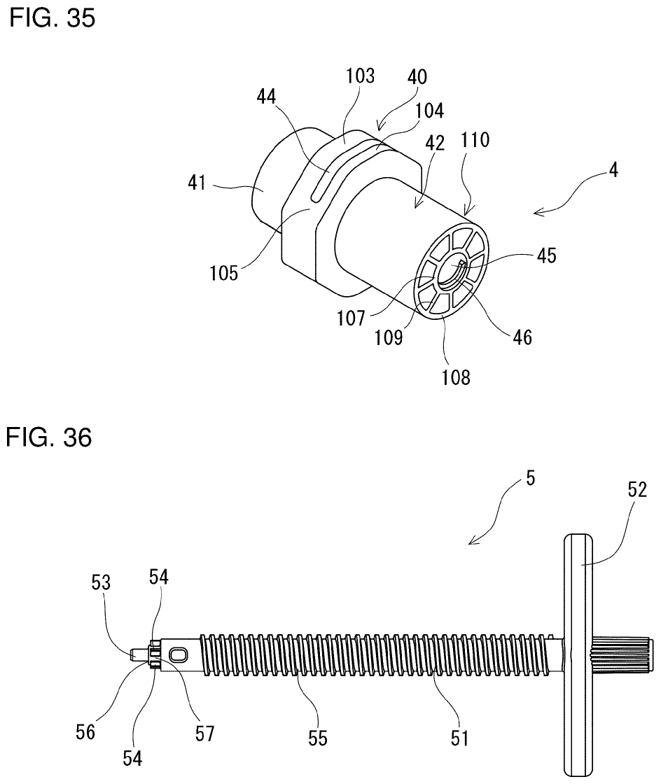

Further, as illustrated in FIG. 35, the insertion portion 110 includes: an inner tubular portion 107 including the insertion-portion-side screwing portion (short spiral rib) 46; an outer tubular portion 108 arranged concentrically with the inner tubular portion 107 on the outer side of the inner tubular portion 107 (in other words, to be separated from an outer peripheral surface of the inner tubular portion 107); and a plurality of connecting plates 109 that connect the outer peripheral surface of the inner tubular portion 107 and an inner peripheral surface of the outer tubular portion 108 and are arranged intermittently in the circumferential direction. It is preferable that four to ten connecting plates 109 be arranged at equal intervals in the circumferential direction of the syringe-mounting member 4. In addition, the connecting plate 109 extends over the entire length of the inner tubular portion 107 in the present embodiment. For this reason, the insertion portion 110, which is a site screwed with the plunger 5, is strongly reinforced, thereby preventing the syringe-mounting member 4 from being distorted at the site. In addition, the syringe-mounting member 4 of the present embodiment has a substantially uniform outer diameter from a distal end of the tubular accommodation portion 42 to a proximal end of the outer tubular portion 108 of the insertion portion 110.

As illustrated in FIGS. 2 to 8, 10, and 36 to 38, the plunger 5 enters the syringe-mounting member 4 on the distal side and can press the gasket pressing assembly 6. Specifically, the plunger 5 includes the shaft portion 51 and the handle 52 provided at the proximal end portion of the shaft portion 51. The shaft portion 51 includes the shaft-side screwing portion 55 provided on the outer surface. In the present embodiment, the shaft-side screwing portion 55 is locked by the spiral groove into which the insertion-portion-side screwing portion 46 (specifically, the short spiral protrusion) of the syringe-mounting member 4 enters such that the screw can be advanced. The distal end portion of the plunger 5 advances in the syringe-mounting member when rotated to one side (rotated forward), and the distal end portion retracts in the syringe-mounting member when rotated to the other side (rotated reversely).

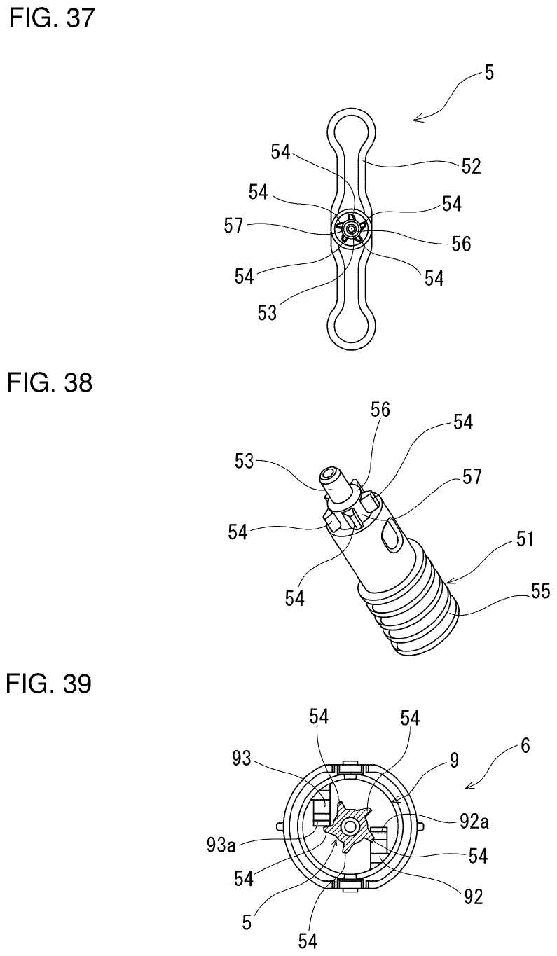

Specifically, as illustrated in FIGS. 37 and 38, a distal base portion 57 that holds the claw-flicking ribs 54 on its outer surface, an annular abutment portion 56 formed by a distal surface of the distal base portion, and a small-diameter cylindrical protruding portion 53 that protrudes from the distal base portion 57 with a diameter smaller than an outer diameter of the abutment portion are provided at the distal end portion of the shaft portion 51. The cylindrical protruding portion 53 can enter the columnar recess 85 (specifically, the cylindrical recess) formed in the central portion of the gasket pressing member 8 and is rotatable after the entry.

An odd number of (three of more) claw-flicking ribs 54 are provided as illustrated in FIGS. 6, 38, and 39, and these claw-flicking ribs 54 are preferably arranged so as to be equiangular with respect to the central axis of the plunger 5. In the present embodiment, five claw-flicking ribs 54 are provided and arranged so as to be equiangular with respect to the central axis of the plunger 5. Further, in the present embodiment, surfaces of the claw-flicking ribs 54 that abut on the claw tip portions 92a and 93a when the plunger 5 is rotated in the screw advancing direction are formed substantially parallel to the central axis of the plunger 5 and further formed as surfaces substantially upright from an outer peripheral surface of the distal base portion 57. Further, a surface of the claw-flicking rib 54 on the side opposite to the upright surface is formed as an inclined surface. For this reason, the claw-flicking rib 54 becomes thicker downward (toward the distal base portion 57).

The handle 52 is formed at the proximal end portion of the shaft portion 51, and is formed by two protruding portions extending in the radial direction from the shaft portion 51. Further, the plunger 5 of the present embodiment has an end portion extending rearward from the handle portion 52.

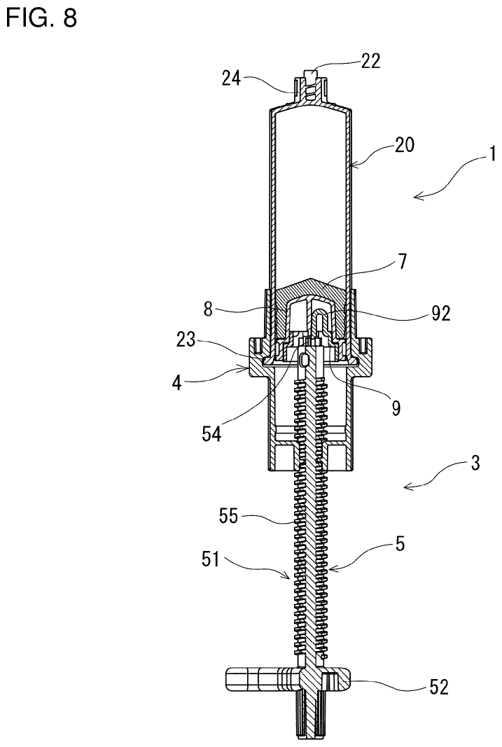

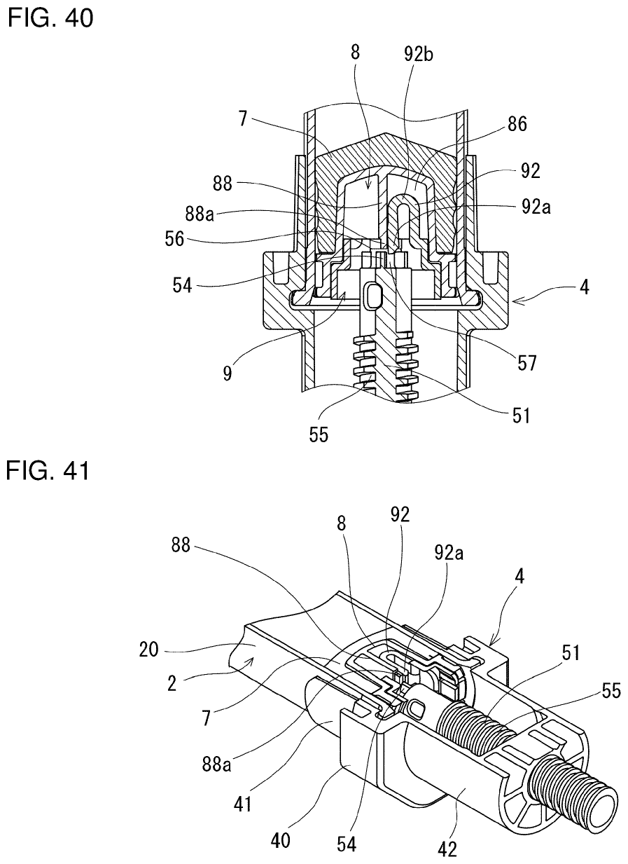

As illustrated in FIG. 8 and FIG. 40, which is an enlarged view of FIG. 8, the claw-flicking ribs 54 are located on the lateral side of the claw tip portions 92a and 93a in a state where the annular abutment portion 56 of the plunger 5 abuts on the plunger abutment portion 62 of the gasket pressing assembly 6 in a pressable manner. Further, as the plunger 5 is rotated, the plunger 5 advances to move the gasket pressing assembly 6 forward, the claw-flicking ribs 54 abut on the claw tip portions 92a and 93a and pass the claw portions 92 and 93 while deforming the claw portions 92 and 93, and further, a sound is generated with abutment of the claw portions 92 and 93 restored after the passage on the wall portions 88 and 89.

Next, operations of the liquid medicine ejection tool 1 and the liquid medicine administration tool according to the present embodiment will be described.

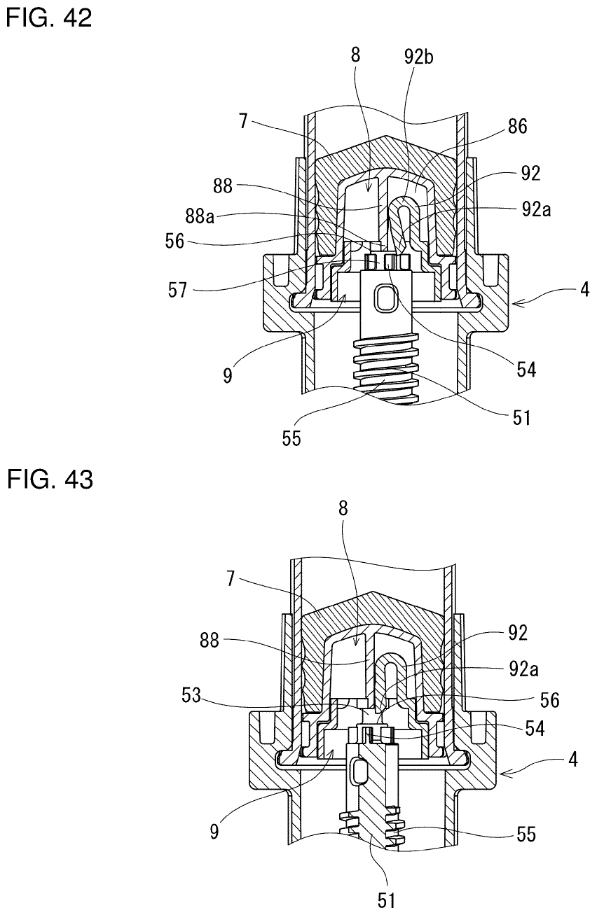

As the plunger is rotated, the state where the annular abutment portion 56 of the plunger 5 abuts on the gasket pressing assembly 6 in a pressable manner, that is, the state illustrated in FIGS. 40 and 41, is formed. In this state, the claw-flicking rib 54 of the plunger 5 is located on the lateral side of each of the claw tip portions 92a and 93a. Then, as the plunger 5 is rotated, the plunger 5 advances, the gasket pressing assembly 6 also advances. As the gasket pressing assembly 6 advances, the gasket 7 also advances. Then, the claw-flicking ribs 54 of the plunger 5 abut on the claw tip portions 92a and 93a of the sound generating member 9, deform the claw portions 92 and 93, and eventually, pass through the claw portions 92 and 93 as illustrated in FIG. 42. Then, after the passage, the claw portions 92 and 93 are restored to the original shapes due to spring properties, and abut on the wall portions 88 and 89 when restored, thereby generating the sound.

In particular, the wall portion 88 is provided with a thin proximal end portion 88a in the illustrated embodiment, and the restored claw tip portion 92a abuts on the thin proximal end portion 88a. Since the proximal end portion 88a is thin, a relatively high-pitched sound is generated. In addition, the two claw portions 92 and 93 and the five claw-flicking ribs 54 are provided in the present embodiment. For this reason, ten collision sounds are generated when the plunger 5 rotates. In other words, the collision sound is generated every time the plunger 5 rotates 36 degrees.

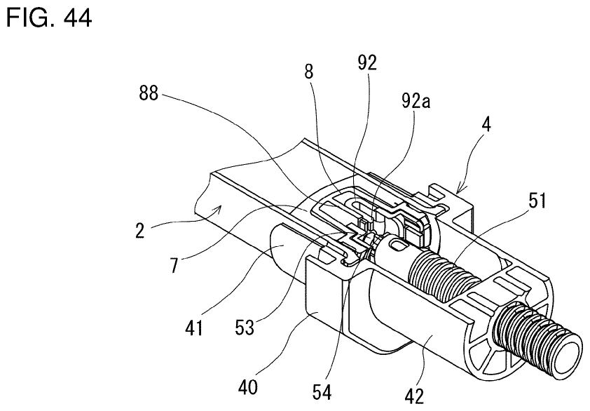

Further, in the gasket pressing tool 3 of the liquid medicine ejection tool 1 of the present embodiment, the claw-flicking rib 54 of the plunger 5 is located on the rear side of each of the claw tip portions 92a and 93a of the sound generating member 9 in the state where the abutment portion 56 of the plunger 5 does not abut on the gasket pressing assembly 6 in a pressable manner as illustrated in FIGS. 43 and 44. For this reason, the claw-flicking ribs 54 do not abut on the claw tip portions 92a and 93a even if the plunger 5 is rotated, and thus, no sound is generated. In addition, the plunger 5 retracts as the plunger 5 is rotated reversely (rotated in the retracting direction) in the state illustrated in FIGS. 40 and 41. No sound is generated even during this reverse rotation. Note that the liquid medicine ejection tool 1 of the present embodiment may be provided in the state where the gasket pressing assembly 6 is mounted on the gasket 7 as illustrated in FIG. 7, in other words, provided with such a state as the initial state. Note that the plunger 5 may or does not necessarily abut on the gasket pressing assembly 6.

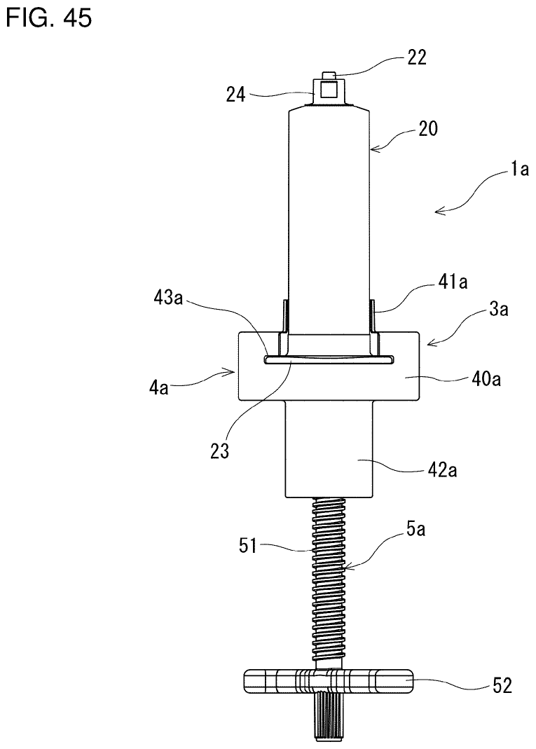

Next, a liquid medicine ejection tool 1a and a gasket pressing tool 3a of the embodiment illustrated in FIGS. 45 to 47 will be described.

The liquid medicine ejection tool 1a and the gasket pressing tool 3a of the present embodiment have the same configuration regarding the sound generation as the liquid medicine ejection tool 1 and the gasket pressing tool 3 described above, and differences therebetween are a form of a syringe-mounting member and a structure of a gasket pressing assembly. The same parts are designated by the same reference signs, and the above description is referred to therefor.

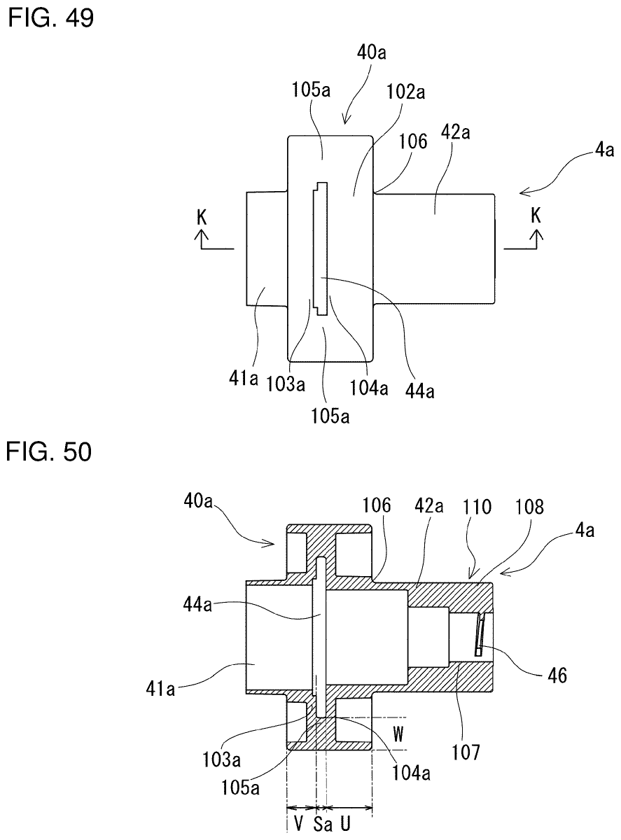

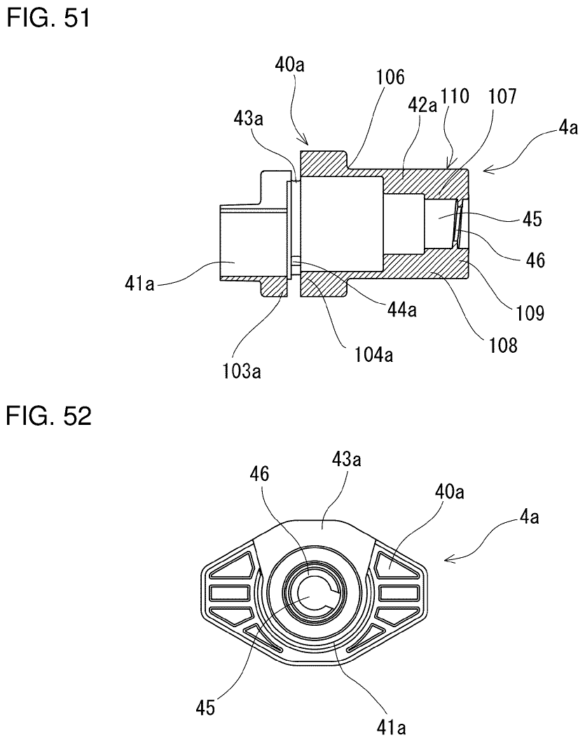

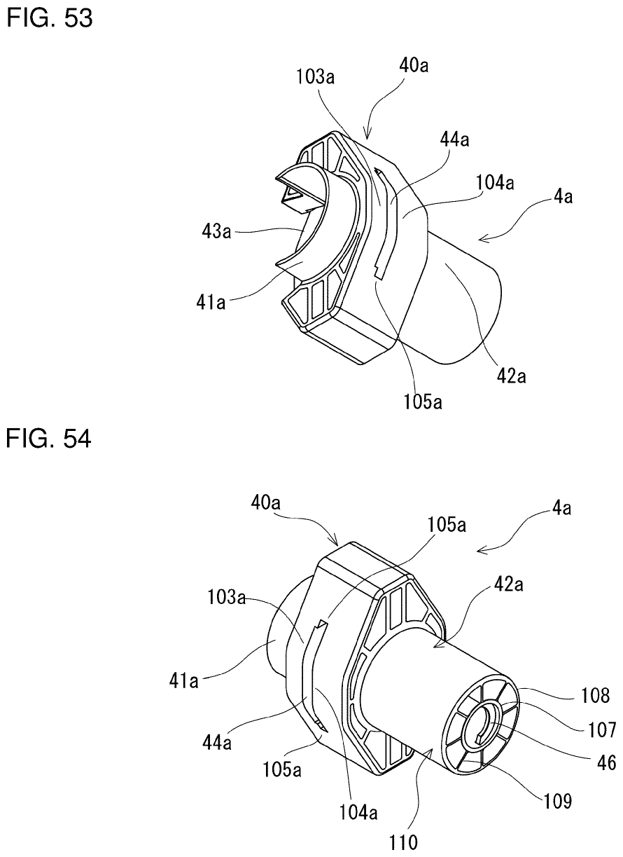

A syringe-mounting member 4a used in the gasket pressing tool 3a of the present embodiment includes a proximal barrel-mounting portion 43a as illustrated in FIGS. 45 to 54. In addition, a gasket pressing assembly 6a is movably accommodated similarly to the above-described embodiment.

The syringe-mounting member 4a used in the gasket pressing tool 3a of the present embodiment includes a body portion (barrel-mounting portion) 40a as illustrated in FIGS. 45 to 53. As illustrated in FIGS. 45 to 54, the gasket pressing tool 3a of the present embodiment movably accommodates the gasket pressing assembly 6a similarly to the one illustrated in FIG. 2.

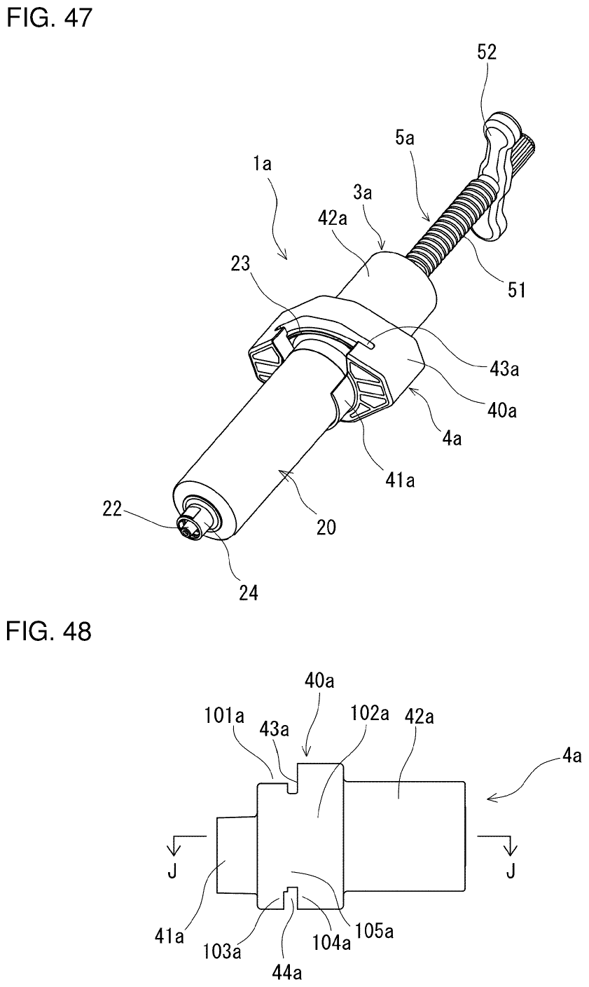

As illustrated in FIGS. 47 to 54, the syringe-mounting member 4a includes: a distal-side support portion 103a, a proximal-side support portion 104a, a connecting portion 105a, a flange accommodation portion 102a, and a flange insertion opening 101a. The distal-side support portion 103a can abut on a distal surface of the flange 23 of the barrel 20 accommodated in the flange accommodation portion 102a, and supports the distal surface of the flange 23.

In the syringe-mounting member 4a of the present embodiment, the distal-side support portion 103a has a predetermined thickness (axial length: V) extending along the central axis of the syringe-mounting member 4a as illustrated in FIG. 50, and the thickness (V) is preferably 5 to 25 mm, and particularly preferably 10 to 15 mm. The distal-side support portion 103a can sufficiently support the distal surface of the flange. The distal-side support portion 103a is preferably capable of supporting 55 to 80% of a distal surface 23a of the flange 23 (in other words, capable of abutting on 55 to 80% of the distal surface 23a), and particularly preferably capable of supporting 70 to 80% (capable of abutting on 70 to 80%) of the distal surface 23a.

In addition, the syringe-mounting member 4a of the present embodiment, the proximal-side support portion 104a has a predetermined thickness (axial length: U) extending along the central axis of the syringe-mounting member 4a as illustrated in FIG. 50, and the thickness (U) is preferably 5 to 25 mm, and particularly preferably 10 to 15 mm. The proximal-side support portion 104a can abut on almost the entire proximal surface 23b of the flange 23. For this reason, the proximal-side support portion 104a can sufficiently support the proximal surface of the flange.

Further, the distal-side support portion 103a and the proximal-side support portion 104a are connected by the connecting portion 105a in the syringe-mounting member 4a of the present embodiment. Further, the connecting portion 105a has a predetermined thickness (axial thickness: Sa) extending along the central axis of the syringe-mounting member 4a as illustrated in FIG. 50, and the thickness (Sa) is preferably −1 to 1 mm with respect to the thickness of the flange 23 of the barrel 20, and particularly preferably −0.5 to 0.5 mm with respect to the thickness of the flange 23 of the barrel 20.

In addition, there are two connecting portions 105a facing each other in the present embodiment as illustrated in FIG. 50, and further, the connecting portion 105a has a radial width W along the radial direction of the syringe-mounting member 4a as illustrated in FIG. 50. The radial width (W) is preferably 1 to 5 mm, and particularly preferably 2 to 3 mm.

The syringe-mounting member 4a includes the proximal barrel-mounting portion 43a that accommodates the flange 23 of the barrel 20 of the syringe 2. In the present embodiment, the proximal barrel-mounting portion 43a is formed using a slot to be capable of inserting the flange 23 of the syringe 2 provided in the body portion 40a from one short-diameter side (in other words, allow horizontal insertion) as illustrated in FIGS. 47 and 50. For this reason, in the state where the syringe 2 is mounted on the syringe-mounting member 4a, one short-diameter portion of the flange 23 and two long-diameter portions facing each other of the syringe 2 are accommodated in the syringe-mounting member 4a, and a short-diameter portion on the other end side is exposed. In addition, an end portion of one short-diameter portion of the flange accommodated in the syringe-mounting member 4a is exposed through a slot opening 44a. In the present embodiment, the proximal barrel-mounting portion 43a is formed by a horizontally elongated U-shaped slot.

In addition, in the present embodiment, the syringe-mounting member 4a includes: a substantially semi-tubular portion (barrel support portion) 41a extending to the distal side from the proximal barrel-mounting portion (flange accommodation portion) 43a to hold the proximal end portion of the mounted syringe 2 as illustrated in FIGS. 48 to 51. Further, the syringe-mounting member 4a includes a tubular accommodation portion 42a extending from the proximal barrel-mounting portion (flange accommodation portion) 43a to the proximal side. The tubular accommodation portion 42a includes the accommodation portion 47 for the gasket pressing assembly therein. Further, the syringe-mounting member 4a includes the lumen 45 extending rearward from the accommodation portion 47, and a protrusion (insertion-portion-side screwing portion) 46 is provided on an inner surface of the lumen 45. The protrusion 46 is a short spiral protrusion.

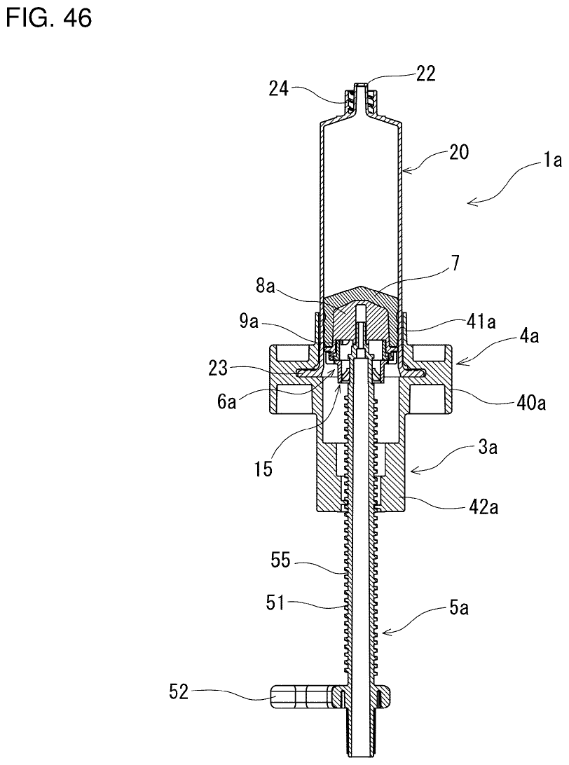

As illustrated in FIGS. 46, 55, and 56, the gasket pressing assembly 6a of the present embodiment includes: a gasket pressing member 8a, a sound generating member 9a, and a joint member 15. As illustrated in FIGS. 46, 55, and 56, the gasket pressing member 8a includes: the gasket-mounting portion 80 that enters the gasket 7 and comes into contact with the inner surface of the gasket; the gasket proximal surface pressing portion 81 formed at a rear portion of the gasket-mounting portion 80; an annular portion 82a extending rearward from the gasket proximal surface pressing portion 81; and an annular rib 66 formed at a proximal end portion of the annular portion 82a. The annular rib 66 is provided with a notch 67. The gasket-mounting portion 80 includes a distal closing portion and a tapered diameter-reducing portion extending from the gasket proximal surface pressing portion 81 to a proximal end of the distal closing portion.

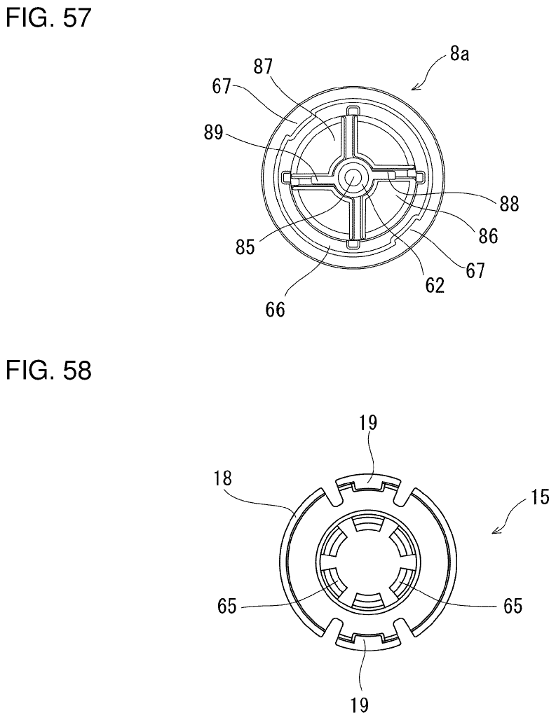

Further, as illustrated in FIG. 57, the gasket pressing member 8a of the present embodiment also includes a sound generating member accommodation portion, the wall portions 88 and 89 extending in the axial direction, the plunger abutment portion 62, and the columnar recess 85 therein and includes a cylindrical portion having the columnar recess 85 and four partition portions extending to the lateral side from the cylindrical portion at the center, which is similar to the above-described embodiment. The internal form of the gasket pressing member 8a is the same as that of the gasket pressing member 8 described above.

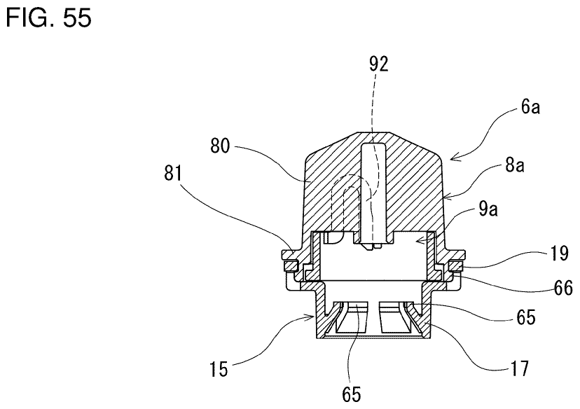

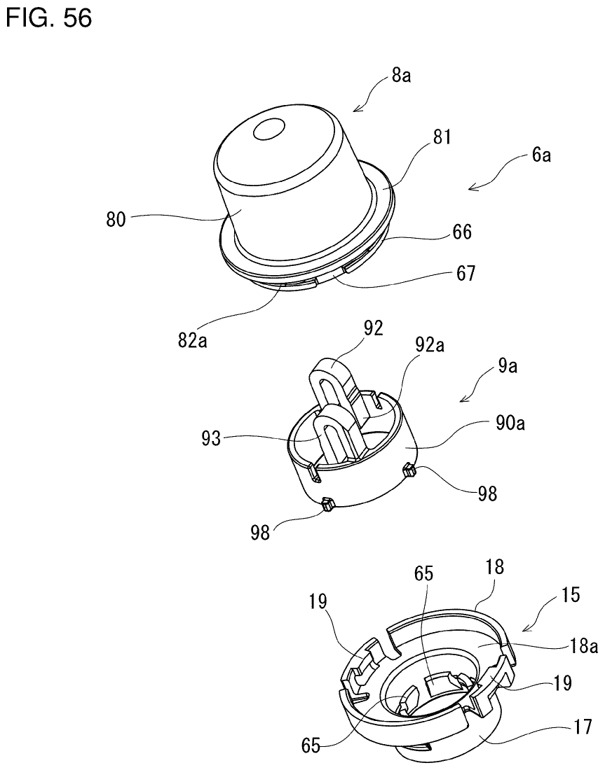

The sound generating member 9a of the present embodiment includes: a tubular body portion 90a; the claw portions 92 and 93 protruding in the distal direction from an inner surface of the tubular body portion 90a; a plurality of protruding portions 98 provided on a lower side surface of the tubular body portion 90a as illustrated in FIGS. 55 and 56. The claw portions 92 and 93 have the same form as that of the sound generating member 9 described above.

In the gasket pressing member 8a of the present embodiment, the gasket pressing member 8a has the same internal form as the gasket pressing member 8 described above, the sound generating member 9a also has the same form as the sound generating member 9 described above, and a sound generating mechanism in the gasket pressing member 8a of the present embodiment is the same as the one described above.

The gasket pressing assembly 6a of the present embodiment includes the joint member 15 arranged at a rear portion of the gasket pressing member 8a. The joint member 15 is mounted on the rear portion of the gasket pressing member 8a that is open and accommodates the sound generating member 9a. The joint member 15 includes a short cylindrical portion 18 having a disc-shaped bottom surface 18a and a small-diameter cylindrical portion 17 extending rearward from the cylindrical portion 18. A rib 19 protruding inward is provided at an upper end of the cylindrical portion 18. The joint member 15 is attached to the gasket pressing member 8a as illustrated in FIG. 55 by being rotated after the rib 19 is inserted into the notch 67 of the annular rib 66 formed at the proximal end of the gasket pressing member 8a. In addition, the joint member 15 includes a plurality of inclined protruding portions 65 extending from an inner surface of the small-diameter cylindrical portion 17 toward the center of the joint member in the distal direction. The plurality of inclined protruding portions 65 are arranged so as to be equiangular with respect to a central axis of the joint member 15.

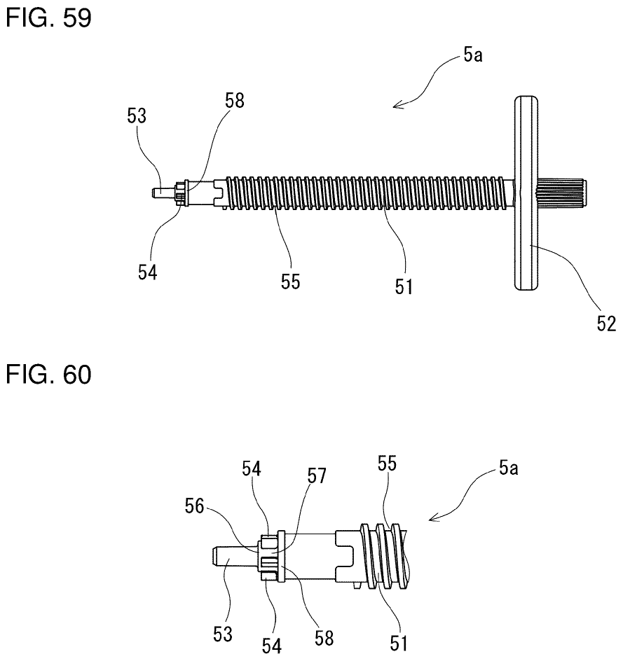

As illustrated in FIGS. 59 and 60, the plunger 5a of the present embodiment has an annular rib 58 which is a distal end portion and is formed on the proximal side of the claw-flicking rib 54. An inner diameter of a portion having the minimum inner diameter, formed by the plurality of inclined protruding portions 65 of the joint member 15, is slightly larger than an outer diameter of the annular rib 58 of the plunger 5a. For this reason, the joint member 15 has a function of preventing detachment of the plunger 5a. In addition, the plunger 5a of the present embodiment includes the claw-flicking ribs 54 and has the same configuration of the distal end portion as the plunger 5 described above. The claw-flicking ribs 54 is located on the lateral side of the claw tip portions 92a and 93a in a state where the annular abutment portion 56 of the plunger 5a abuts on the gasket pressing assembly 6a in a pressable manner. Further, as the plunger 5a is rotated, the plunger 5a advances to move the gasket pressing assembly 6a forward, the claw-flicking ribs 54 abut on the claw tip portions 92a and 93a and pass the claw portions 92 and 93 while deforming the claw portions 92 and 93, and further, a sound is generated with abutment of the claw portions 92 and 93 restored after the passage on the wall portions 88 and 89.

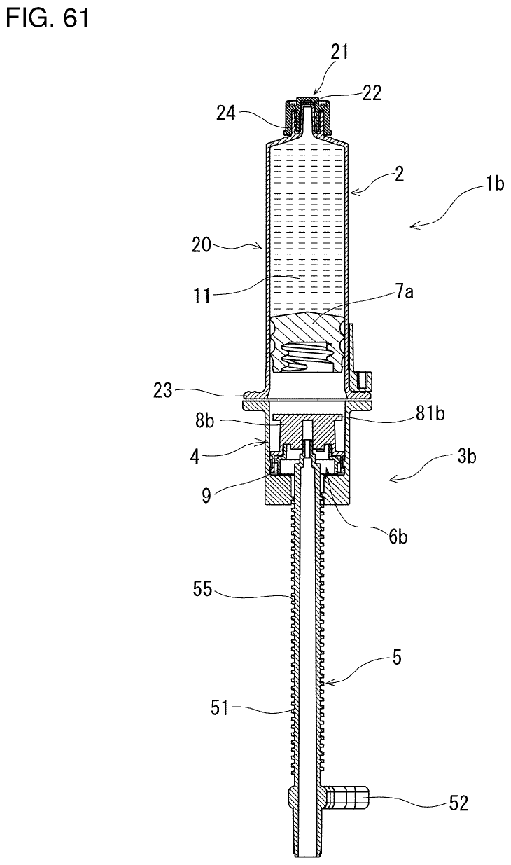

Note that a gasket pressing member may be of a type including a liquid medicine administration tool 1b and a gasket pressing tool 3b for a syringe according to an embodiment illustrated in FIG. 61 in part of the above-described embodiments. A gasket pressing member 8b of the present embodiment does not practically have a portion that enters the gasket 7a. For this reason, a gasket pressing assembly 6b also does not practically have a portion that enters the gasket 7a. Further, the gasket pressing member 8b includes a pressing portion 81b, which can abut on and press a rear end surface of the gasket 7a, at its distal end portion. Note that the liquid medicine administration tool 1b and the gasket pressing tool 3b for a syringe according to the present embodiment also have the same sound generating mechanism as the liquid medicine administration tools 1 and 1a and the gasket pressing tools 3 and 3a for the syringe described above.

Next, a case of performing endoscopic submucosal dissection (ESD) or endoscopic mucosal resection (EMR) will be described as an example of a treatment method using the liquid medicine administration tool (liquid medicine ejection tool 1). First, the endoscopic submucosal dissection will be described. This procedure is usually performed by two operators.

A first operator injects a sodium hyaluronate solution, and a second operator performs the excision of an affected site for confirmation of an endoscopic image.

The first operator prepares the liquid medicine administration tool (liquid medicine ejection tool 1) in which the gasket pressing tool 3 is mounted on the prefilled syringe 2 filled with a sodium hyaluronate solution 11. When the prepared liquid medicine administration tool is in the state illustrated in FIGS. 1 and 2, the plunger 5 is rotated to mount the gasket pressing assembly 6 in the gasket 7 as illustrated in FIG. 7. Then, the sealing cap 21 is removed from the prefilled syringe 2.

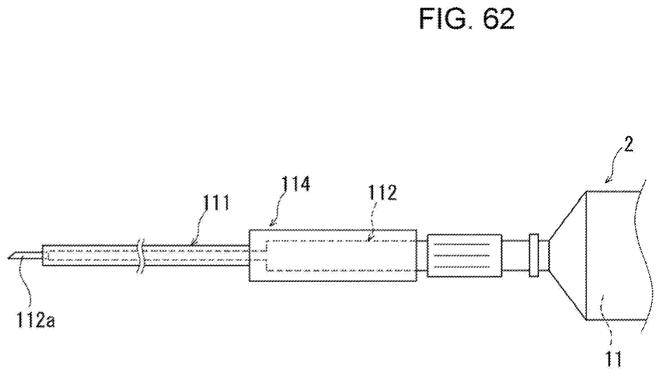

Next, as illustrated in FIG. 62, the first operator connects a distal end portion of the syringe 2 to a proximal end portion of an inner catheter 112 of a puncture device 111 and causes a needle 112a to protrude from an outer catheter 114. In this state, the plunger 5 is rotated to perform priming to fill the inner catheter 112 and a lumen of the needle 112a with the sodium hyaluronate solution 11. Note that the inner catheter 112 of the puncture device 111 is slidable in the axial direction within a regulated range with respect to the outer catheter 114. The needle 112a is connected to a distal end of the inner catheter 112. When the inner catheter 112 is at a retracted position relative to the outer catheter 114, the needle 112a is accommodated in the outer catheter 114. When the inner catheter 112 is at an advanced position relative to the outer catheter 144, the needle 112a protrudes from a distal end of the outer catheter 114.

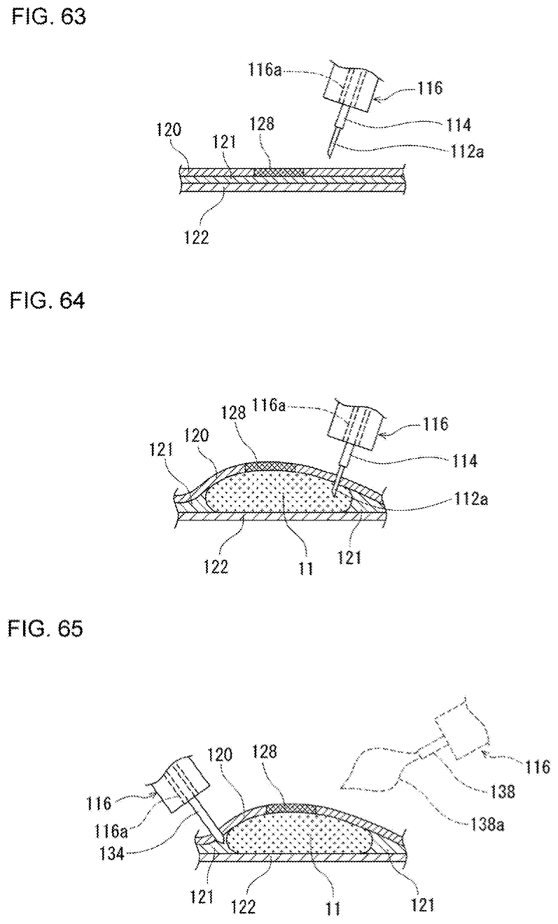

Next, the second operator accommodates the needle 112a in the outer catheter 114. Then, the puncture device 111 is inserted into an insertion hole (forceps hole) 116a (see FIG. 63) of an endoscope 116. Subsequently, the second operator causes the needle 112a to protrude from a distal end of the endoscope 116 in a digestive tract of a patient as illustrated in FIG. 63. Then, the needle 112a penetrates a mucosa 120 around a lesion 128 to reach a submucosa 121. The second operator informs the first operator that the needle has reached the submucosa 121.

The second operator informs the first operator of the number of click sounds for injection. The first operator rotates the plunger 5 by the number of informed click sounds to inject the sodium hyaluronate solution 11 into the submucosa 121. The second operator visually confirms that a predetermined amount of the sodium hyaluronate solution 11 has been injected by confirming the number of click sounds. The second operator confirms the endoscopic image and, if necessary, informs the first operator of the number of click sounds for the maximum injection. The first operator rotates the plunger 5 by the number of informed click sounds to inject the sodium hyaluronate solution 11 into the submucosa 121. As this operation is performed a required number of times to raise the lesion 128 to a target size, the state illustrated in FIG. 64 is obtained. In this state, the submucosa 121 below the lesion 128 is turned into a state of being separated from a muscle layer 122.

The second operator notifies the first operator that the injection operation of the sodium hyaluronate solution has been completed. The first operator who has been notified reversely rotates the plunger 5 to release the pressure. As a result, the ejection of the sodium hyaluronate solution 11 from the syringe 2 is stopped.

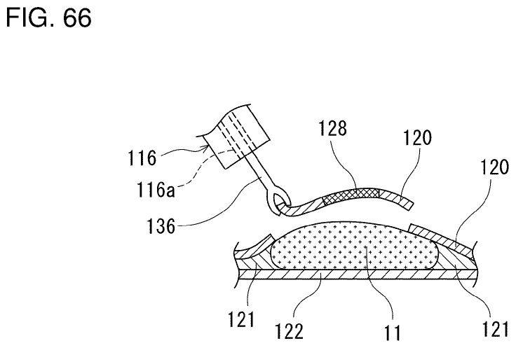

Next, the second operator inserts a peeling device 134 (needle knife or the like) into the insertion hole (forceps hole) 116a of the endoscope 116 as illustrated in FIG. 65 to cut the mucosa 120 around the lesion 128 using the peeling device 134. Subsequently, the second operator inserts a grasping device 136 into the insertion hole 116a of the endoscope 116 and peels the mucosa 120 having the lesion 128 and taken the peeled mucosa 120 out of the body using the grasping device 136 as illustrated in FIG. 66.

On the other hand, in the case of performing the endoscopic mucosal resection, a cutting device 138 illustrated by the virtual line in FIG. 65 is used, instead of peeling the mucosa 120 with the peeling device 134 described above. The cutting device 138 has a snare ring 138a (loop-shaped electric knife). The second operator causes the snare ring 138a to protrude from the endoscope 116 inside the patient's body and hooks the snare ring 138a onto the mucosa 120 around the raised lesion 128. Then, the snare ring 138a is narrowed, a high-frequency current is caused to flow through the snare ring 138a so that the mucosa 120 having the lesion 128 is cut. The cut mucosa 120 is collected by the grasping device 136 illustrated in FIG. 66. Other procedure steps are performed in the same manner as the above-described endoscopic submucosal dissection.

### INDUSTRIAL APPLICABILITY

A liquid medicine ejection tool for solving a first problem is configured as follows.

(1) A liquid medicine ejection tool provided with: a syringe that includes a barrel having a flange at a proximal end portion and a gasket slidable inside the barrel and made of an elastic material; and a gasket pressing tool used by being mounted on the syringe, wherein

the gasket includes a tubular portion, a distal closing portion that closes a distal end of the tubular portion, a lumen portion defined by the tubular portion and the distal closing portion, and an annular rib provided on an outer surface of the tubular portion,

the gasket pressing tool includes: a gasket pressing member having a gasket-mounting portion, which is capable of entering the lumen portion of the gasket and made of a material harder than the gasket, and a gasket proximal surface pressing portion provided at a proximal end portion of the gasket-mounting portion; a syringe-mounting member that movably accommodates the gasket pressing member and has a proximal barrel-mounting portion that is mountable to a proximal end portion of the barrel having the flange; and a plunger that has a distal end portion entering the syringe-mounting member and is capable of pressing the gasket pressing member in a distal direction,

the gasket has an original lumen portion axial length S when the gasket is not inserted into the barrel and the gasket pressing member is not mounted, the gasket-mounting portion of the gasket pressing member has an axial length T, and the axial length T is longer than the original lumen portion axial length S, and

when the gasket is inserted into the barrel and the gasket pressing member is mounted, a mounting portion distal surface of the gasket-mounting portion of the gasket pressing member abuts on a lumen distal surface of the lumen portion of the gasket, and the gasket proximal surface pressing portion of the gasket pressing member becomes close to or abuts on a proximal surface of the gasket.

The liquid medicine ejection tool which solves the first problem is provided with: the syringe including the barrel having the flange at the proximal end portion and the gasket slidable in the barrel and made of the elastic material; and the gasket pressing tool used by being mounted on the syringe. The gasket includes a tubular portion, a distal closing portion closing a distal end of the tubular portion, a lumen portion defined by the tubular portion and the distal closing portion, and an annular rib provided on an outer surface of the tubular portion. The gasket pressing tool includes: a gasket pressing member having a gasket-mounting portion, which can enter the lumen portion of the gasket and made of a material harder than the gasket, and a gasket proximal surface pressing portion provided at its proximal end portion of the gasket-mounting portion; a syringe-mounting member that movably accommodates the gasket pressing member and has a proximal barrel-mounting portion which can be mounted to a proximal end portion of the barrel having the flange; and a plunger that has a distal end portion entering the syringe-mounting member and can press the gasket pressing member in a distal direction. The gasket has the original lumen portion axial length S when the gasket is not inserted into the barrel and the gasket pressing member is not mounted, the gasket-mounting portion of the gasket pressing member has the axial length T, and the axial length T is longer than the original lumen portion axial length S. When the gasket is inserted into the barrel and the gasket pressing member is mounted, the mounting portion distal surface of the gasket-mounting portion of the gasket pressing member abuts on the lumen distal surface of the lumen portion of the gasket, and the gasket proximal surface pressing portion of the gasket pressing member becomes close to or abuts on the proximal surface of the gasket.

In this liquid medicine ejection tool, the gasket is practically in a state where the lumen portion is not deformable, and even if the gasket is elastically deformed when an ejection operation of a liquid medicine is started, there is no volume change. Accordingly, even if the liquid medicine is a highly viscous substance, a time lag between an administration operation and actual outflow of the highly viscous substance is extremely short, and it is possible to administer the highly viscous substance to a target site in a timely and favorable manner.

In addition, the above-described embodiment for solving the first problem may be configured as follows.

(2) The liquid medicine ejection tool according to (1) above, wherein the axial length T is 1.05 to 1.20 times of the original lumen portion axial length S.

(3) The liquid medicine ejection tool according to (1) or (2) above, wherein the gasket-mounting portion includes: a distal end portion having a distal surface of the mounting portion; and a proximal-side tapered portion which extends from the distal end portion of the gasket-mounting portion to the gasket proximal surface pressing portion and is enlarged in diameter toward the rear side to have a tapered shape, and the entire outer surface of the proximal-side tapered portion becomes close to or abuts on an inner surface of the lumen portion of the gasket.

(4) The liquid medicine ejection tool according to (3) above, wherein a taper angle of the proximal-side tapered portion of the gasket-mounting portion is 1 to 8 degrees.

(5) The liquid medicine ejection tool according to any one of (1) to (4) above, wherein the entire outer surface of the gasket-mounting portion has a shape that substantially coincides with the entire inner surface of the lumen portion of the gasket when being inserted into the barrel.

(6) The liquid medicine ejection tool according to any one of (1) to (5) above, wherein

the lumen distal surface of the lumen portion of the gasket has: a flat central flat surface at the center of the lumen distal surface; and a lumen portion tapered surface that extends rearward and radially outward from an outer edge of the central flat surface,

the distal closing portion of the gasket has: a tapered gasket distal surface whose diameter is reduced toward a distal end; and a uniform-thickness portion which is sandwiched between the gasket distal surface and the lumen portion tapered surface and has a substantially uniform thickness,

the mounting portion distal surface of the gasket-mounting portion has: a flat distal surface, which is flat, at the center of the mounting portion distal surface; and a tapered outer edge surface extending rearward and radially outward from an outer edge of the flat distal surface, and

when the gasket is inserted into the barrel and the gasket pressing member is mounted, the flat distal surface of the gasket-mounting portion abuts on the central flat surface of the lumen portion of the gasket, and the tapered outer edge surface of the gasket-mounting portion becomes close to or abuts on the lumen portion tapered surface of the lumen portion of the gasket.

(7) The liquid medicine ejection tool according to any one of (1) to (6) above, wherein the gasket has an original axial length Q and an original lumen portion axial length S in the state where the gasket is not inserted into the syringe and the gasket pressing member is not mounted, the gasket has a deformed axial length P and a deformed lumen portion axial length R in the state of being inserted into the syringe and having the gasket pressing member mounted thereon, the deformed axial length P is longer than the original axial length Q, and the deformed lumen portion axial length R is longer than the original lumen portion axial length S.

(8) The liquid medicine ejection tool according to any one of (1) to (7) above, wherein the gasket pressing member includes a plunger abutment portion that is capable of abutting on and being separated from the distal end portion of the plunger.

(9) The liquid medicine ejection tool according to (8) above, wherein in the state where the gasket is inserted into the barrel and the gasket pressing member is mounted, the distal end portion of the plunger abuts on the plunger abutment portion of the gasket pressing member when the plunger is advanced in the distal direction, and the mounting state of the gasket pressing member on the gasket is maintained and the plunger abutment portion is separated from the distal end portion of the plunger when the plunger is pulled in the proximal direction.

(10) The liquid medicine ejection tool according to (9) above, wherein when the plunger is pulled in the proximal direction in the state where the gasket is inserted into the barrel and the gasket-mounting portion is inserted into the lumen portion of the gasket, the mounting portion distal surface of the gasket-mounting portion maintains a state of abutting on the lumen distal surface of the lumen portion of the gasket.

(11) The liquid medicine ejection tool according to any one of (1) to (10) above, wherein the syringe-mounting member includes a tubular accommodation portion that extends in the proximal direction from a body portion having the proximal barrel-mounting portion and is capable of accommodating the gasket-mounting portion.

(12) The liquid medicine ejection tool according to (11) above, wherein

the plunger includes: a shaft portion which extends in the proximal direction from the distal end portion and has a shaft-side screwing portion on an outer peripheral surface; and a handle configured to rotate the plunger provided at a proximal end portion of the shaft portion,

the syringe-mounting member includes a tubular insertion portion which extends in the proximal direction from the tubular accommodation portion and in which the shaft portion of the plunger is inserted,

the insertion portion has an insertion-portion-side screwing portion, which is screwed with the shaft-side screwing portion of the shaft portion, on an inner surface, and

the plunger is advanced in the distal direction by screwing between the shaft-side screwing portion and the insertion-portion-side screwing portion along with the rotation of the shaft portion.

A liquid medicine administration tool for solving the first problem is configured as follows.

(13) A liquid medicine administration tool including: the liquid medicine ejection tool according to any one of (1) to (12) above; a liquid medicine charged in the syringe; and a sealing member sealing the distal end portion of the barrel.

The liquid medicine administration tool includes: the above-described liquid medicine ejection tool; the liquid medicine charged in the syringe; and the sealing member sealing the distal end portion of the barrel. For this reason, there is no volume change even if the gasket is elastically deformed after the ejection of the liquid medicine is started, which is similar to the above description. Accordingly, even if the liquid medicine is a highly viscous substance, a time lag between an administration operation and actual outflow of the highly viscous substance is extremely short, and it is possible to administer the highly viscous substance to a target site in a timely and favorable manner.

A gasket pressing tool for solving a second problem is configured as follows.

(14-1) A gasket pressing tool used by being mounted on a syringe, which includes: a barrel having an annular flange at a proximal end portion; and a gasket that is slidable in the barrel and is made of an elastic material,

the gasket pressing tool including: a gasket pressing member configured to press the gasket; a tubular syringe-mounting member that movably accommodates the gasket pressing member and is mountable on the proximal end portion of the barrel; and a plunger which has a distal end portion entering the syringe-mounting member and is capable of pressing the gasket pressing member in a distal direction,

wherein the plunger includes: the distal end portion capable of pressing the gasket pressing member; a shaft portion that extends in a proximal direction from the distal end portion and has a shaft-side screwing portion on an outer peripheral surface; and a handle configured to rotate the plunger provided at a proximal end portion of the shaft portion,

the syringe-mounting member includes: a body portion capable of accommodating the flange of the barrel; a tubular accommodation portion which extends from a proximal end of the body portion in a proximal direction of the gasket pressing tool and is capable of accommodating the gasket pressing member; and a tubular insertion portion which extends from a proximal end of the tubular accommodation portion in the proximal direction of the gasket pressing tool and in which the shaft portion of the plunger is inserted,

the insertion portion of the syringe-mounting member has an insertion-portion-side screwing portion, which is screwed with the shaft-side screwing portion of the shaft portion, on an inner peripheral surface,

the body portion includes: a distal-side support portion capable of supporting a distal surface of the flange; a proximal-side support portion capable of supporting a proximal surface of the flange; a connecting portion connecting the distal-side support portion and the proximal-side support portion; a flange accommodation portion which is formed of the distal-side support portion, the proximal-side support portion, and the connecting portion, and is capable of accommodating the flange; and a flange insertion opening which is open to a lateral side of the body portion and is configured to insert the flange into the flange accommodation portion, and

the distal-side support portion has an axial thickness of 5 to 25 mm along a central axis of the syringe-mounting member, the proximal-side support portion has an axial thickness of 5 to 25 mm along the central axis of the syringe-mounting member, and the connecting portion has a radial width of 1 to 5 mm along a radial direction of the syringe-mounting member.

The gasket pressing tool for solving the second problem is used by being mounted on the syringe which includes: the barrel having the annular flange at the proximal end portion; and the gasket slidable in the barrel and made of the elastic material. The gasket pressing tool includes: the gasket pressing member configured to press the gasket; the tubular syringe-mounting member that movably accommodates the gasket pressing member and can be mounted on the proximal end portion of the barrel; and the plunger that has the distal end portion entering the syringe-mounting member and can press the gasket pressing member in the distal direction. The plunger includes: the distal end portion that can press the gasket pressing member; the shaft portion which extends from the distal end portion in the proximal direction and has the shaft-side screwing portion on the outer peripheral surface; and the handle configured to rotate the plunger provided at the proximal end portion of the shaft portion. The syringe-mounting member includes: the body portion (barrel-mounting portion) which can accommodate the flange of the barrel; the tubular accommodation portion which extends from a proximal end of the body portion in the proximal direction of the gasket pressing tool and can accommodate the gasket pressing member; and the tubular insertion portion which extends from the proximal end of the tubular accommodation portion in the proximal direction of the gasket pressing tool and into which the shaft portion of the plunger is inserted. The insertion portion of the syringe-mounting member has the insertion-portion-side screwing portion, which is screwed with the shaft-side screwing portion of the shaft portion, on the inner peripheral surface, and the body portion of the syringe-mounting member includes: the distal-side support portion capable of supporting the distal surface of the flange; the proximal-side support portion capable of supporting the proximal surface of the flange; the connecting portion connecting the distal-side support portion and the proximal-side support portion; the flange accommodation portion which is formed of the distal-side support portion, the proximal-side support portion, and the connecting portion, and is capable of accommodating the flange; and the flange insertion opening which is open to a lateral side of the body portion and is configured to insert the flange into the flange accommodation portion. The distal-side support portion has the axial thickness of 5 to 25 mm along the central axis of the syringe-mounting member, the proximal-side support portion has the axial thickness of 5 to 25 mm along the central axis of the syringe-mounting member, and the connecting portion has the radial width of 1 to 5 mm along the radial direction of the syringe-mounting member.

In this gasket pressing tool (medicine ejection tool), the body portion of the syringe-mounting member includes the flange accommodation portion including: the distal-side support portion having the above thickness; the proximal-side support portion having the above thickness; and the connecting portion which connects the distal-side support portion and the proximal-side support portion and has the above thickness, and thus, the deformation that spreads in the connecting portion is extremely rare even if a rearward pressing force is applied. For this reason, there is a short time lag between an administration operation of a highly viscous substance and actual outflow of the substance to be administered, and the substance to be administered can be administered to a target site in a timely and favorable manner.

In addition, the above-described embodiment for solving the second problem may be configured as follows.

(14-2) The gasket pressing tool according to (14-1) above, where the body portion of the syringe-mounting member has an outer peripheral surface which extends along the central axis of the syringe-mounting member from a distal end of the distal-side support portion to a proximal end of the proximal-side support portion.

(14-3) The gasket pressing tool according to (14-1) or (14-2) above, wherein the distal-side support portion can support 55 to 80% of the distal surface of the flange.

(14-4) The gasket pressing tool according to any one of (14-1) to (14-3) above, wherein the body portion of the syringe-mounting member has an opening which faces the flange insertion opening with respect to the central axis of the syringe-mounting member and allows entry of a part of the flange accommodated in the flange accommodation portion.

(14-5) The gasket pressing tool according to any one of (14-1) to (14-4) above, wherein the insertion portion of the syringe-mounting member includes: an inner tubular portion having the insertion-portion-side screwing portion on an inner peripheral surface; an outer tubular portion arranged concentrically with the inner tubular portion outside the inner tubular portion; and a plurality of connecting plates which connect the outer peripheral surface of the inner tubular portion and the inner peripheral surface of the outer tubular portion and are arranged intermittently in a circumferential direction.

(14-6) The gasket pressing tool according to (14-5) above, wherein, as the plurality of connecting plates, four to ten connecting plates are arranged at equal intervals in the circumferential direction of the syringe-mounting member.

(14-7) The gasket pressing tool according to (14-6) above, wherein the syringe-mounting member has a substantially uniform outer diameter from a distal end of the tubular accommodation portion to a proximal end of the outer tubular portion of the insertion portion.

(14-8) The gasket pressing tool according to any one of (14-1) to (14-7) above, wherein the tubular accommodation portion has an outer diameter smaller than an outer diameter of the proximal-side support portion of the body portion of the syringe-mounting member.

(14-9) The gasket pressing tool described in (14-8) above, wherein the tubular accommodation portion has a curved portion which is enlarged in diameter toward a distal end and is connected to the proximal end of the proximal-side support portion of the body portion of the syringe-mounting member.

(14-10) The gasket pressing tool according to (14-9) above, wherein the curved portion has a radius of curvature of 0.3 to 3.

(14-11) The gasket pressing tool according to any one of (14-1) to (14-10) above, wherein the syringe-mounting member includes a barrel support portion which extends in the distal direction from the flange accommodation portion and supports an outer peripheral surface of a tubular portion of the barrel.

A liquid medicine administration tool for solving the second problem is configured as follows.

(15) A liquid medicine administration tool including: the gasket pressing tool according to any one of (14-1) to (14-11) above; the syringe; a liquid medicine charged in the syringe; and a sealing member sealing the distal end portion of the barrel.

The liquid medicine administration tool which solves the second problem includes: the above gasket pressing tool; the syringe, the liquid medicine charged in the syringe, and the sealing member sealing the distal end portion of the barrel.

For this reason, the liquid medicine administration tool uses the above-described gasket pressing tool, and thus, there is a short time lag between an administration operation of a highly viscous substance and actual outflow of the substance to be administered, and the substance to be administered can be administered to a target site in a timely and favorable manner.

A gasket pressing tool for solving a third problem is configured as follows.

(16-1) A gasket pressing tool for a syringe, which is used by being mounted on a syringe having a barrel and a gasket slidable in the barrel,

the gasket pressing tool including: a gasket pressing assembly which includes a gasket pressing member and a sound generating member; a syringe-mounting member which has a syringe-mounting portion and movably accommodates the gasket pressing assembly; and a plunger which enters the syringe-mounting member on a distal side and is capable of pressing the gasket pressing assembly by rotation,

wherein the sound generating member includes a claw portion which is elastically deformable, the gasket pressing member includes a wall portion abutting on the claw portion, and the claw portion includes a claw tip portion protruding from the wall portion,

the plunger includes a claw-flicking rib provided on a side surface of the distal end portion,

the claw-flicking rib is located on a rear side of the claw tip portion in a state where the plunger does not abut on the gasket pressing assembly in a pressable manner, and the claw-flicking rib is located on a lateral side of the claw tip portion in a state where the plunger abuts on the gasket pressing assembly in a pressable manner, and

as the plunger is rotated, the plunger advances to move the gasket pressing assembly forward, the claw-flicking rib abuts on the claw tip portion and passes the claw portion while deforming the claw portion, and a sound is generated as the claw portion restored after the passage abuts on the wall portion.

The gasket pressing tool for a syringe of that solves the third problem is used by being mounted on a syringe having the barrel and the gasket slidable in the barrel. The gasket pressing tool for the syringe includes: the gasket pressing assembly which includes the gasket pressing member and the sound generating member mounted on the gasket pressing member; the syringe-mounting member which has the syringe-mounting portion and movably accommodates the gasket pressing assembly; and the plunger which enters the syringe-mounting member on the distal side and is capable of pressing the gasket pressing assembly by rotation. The sound generating member includes the claw portion which is elastically deformable, the gasket pressing member includes the wall portion abutting on the claw portion, the claw portion includes the claw tip portion protruding from the wall portion, and the plunger includes the claw-flicking rib provided on the side surface of the distal end portion. In the state where the plunger does not abut on the gasket pressing assembly in a pressable manner, the claw-flicking rib is located on the rear side the claw tip portion. In the state where the plunger abuts on the gasket pressing assembly in a pressable manner, the claw-flicking rib is located on the lateral side of the claw tip portion. As the plunger is rotated, the plunger advances to advance the gasket pressing assembly, and the claw-flicking rib abuts on the claw tip portion, passes the claw portion while deforming the claw portion. The sound is generated as the claw portion restored after the passage abuts on the wall portion.

In this gasket pressing tool for the syringe, if the plunger is rotated in the advancing direction in the state where the plunger abuts on the gasket pressing assembly in a pressable manner, the claw-flicking rib provided at the distal end portion flicks the claw tip portion of the sound generating member, and the flicked claw tip portion returns by a restoring force after elastic deformation to collide with the wall portion of the gasket pressing member. Then, a collision sound is generated at the time of collision. A certain magnitude of the collision sound is generated regardless of the rotation speed of the plunger, and is reliably generated even when the plunger is slowly rotated. In addition, in the state where the plunger does not abut on the gasket pressing assembly in a pressable manner, the claw-flicking rib is located on the rear side of the claw tip portion, and thus, the claw-flicking rib does not abut on the claw tip portion in such a state even if the plunger is rotated, and thus, no sound is generated.

In addition, the above-described embodiment for solving the third problem may be configured as follows.

(16-2) The gasket pressing tool for a syringe according to (16-1) above, wherein the gasket pressing member includes: a body portion that can enter the gasket; and a flange provided at a rear end portion; a sound generating member accommodation portion, the wall portion extending in an axial direction; an engagement portion with respect to the sound generating member; and a plunger abutment portion.

(16-3) The gasket pressing tool for a syringe according to (16-1) or (16-2) above, wherein the sound generating member includes a tubular body portion, and the claw portion protruding in a distal direction from an inner surface of the tubular body portion, and the claw portion includes a base portion fixed to the inner surface of the tubular body portion, a curved portion protruding forward from the base portion, and the claw tip portion facing a rear end direction.

(16-4) The gasket pressing tool for a syringe according to any one of (16-1) to (16-3) above, wherein the claw tip portion is tapered toward a distal end.

(16-5) The gasket pressing tool for a syringe according to any one of (16-1) to (16-4) above, wherein the number of the claw portions is two, and the two claw tip portions substantially face each other.

(16-6) The gasket pressing tool for a syringe according to any one of (16-1) to (16-5) above, wherein the plunger includes: a shaft portion; a distal base portion having the claw-flicking rib provided at a distal end portion of the shaft portion; a cylindrical protruding portion protruding from the distal base portion; and a handle portion provided at a rear end portion of the shaft portion.

(16-7) The gasket pressing tool for a syringe according to any one of (16-1) to (16-6) above, wherein a plurality of the claw-flicking ribs are provided.

(16-8) The gasket pressing tool for a syringe according to any one of (16-1) to (16-6) above, wherein an odd number of the claw-flicking ribs are provided, the odd number being three or more, and the claw-flicking ribs are arranged so as to be equiangular with respect to a central axis of the plunger.

(16-9) The gasket pressing tool for a syringe according to any one of (16-1) to (16-8) above, wherein the claw-flicking rib is provided with a substantially upright surface that abuts on the claw tip portion when the plunger rotates in a screw advancing direction.

(16-10) The gasket pressing tool for a syringe according to any one of (16-1) to (16-9) above, wherein the syringe-mounting portion includes a flange accommodation portion capable of accommodating a proximal end portion of the syringe provided with the flange.

(16-11) The gasket pressing tool for a syringe according to (16-10) above, wherein the syringe-mounting member includes a lumen located on a rear end side of the flange accommodation portion, and a syringe-mounting-member-side screwing portion on an inner surface of the lumen, and the plunger includes a shaft-side screwing portion, which is screwed with the syringe-mounting-member-side screwing portion, on an outer surface of the shaft portion.

(16-12) The gasket pressing tool for a syringes according to (16-11) above, wherein the syringe-mounting-member-side screwing portion is a short spiral protrusion, and the shaft-side screwing portion is a spiral groove into which the spiral protrusion is inserted to be screwable.

A liquid medicine administration tool for solving the third problem is configured as follows.

(17) A liquid medicine administration tool including: the gasket pressing tool for a syringe according to any one of (16-1) to (16-12) above; and a prefilled syringe that includes a barrel, a liquid medicine charged in the barrel, and the gasket slidably accommodated in the barrel, wherein the prefilled syringe is mounted on the syringe-mounting portion of the gasket pressing tool for a syringe.

The liquid medicine administration tool which solves the third problem includes: the above-described gasket pressing tool for a syringe; and the prefilled syringe that includes the barrel, the liquid medicine charged in the barrel, and the gasket slidably accommodated in the barrel, and the prefilled syringe is mounted on the syringe-mounting portion of the gasket pressing tool for a syringe.

According to this liquid medicine administration tool, the amount of rotation of the plunger and a dose of the liquid medicine can be easily grasped by a generated sound.

The detailed description above describes embodiments of a medicine administration tool and a gasket pressing tool representing examples of the medicine administration tool and gasket pressing tool disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A liquid medicine ejection tool comprising:
a syringe that includes a barrel having a flange at a proximal end portion of the barrel, and a gasket that is slidable inside the barrel and is made of an elastic material; and
a gasket pressing tool that is mountable on the syringe, wherein
the gasket includes a tubular portion, a distal closing portion that closes a distal end of the tubular portion, a lumen portion defined by the tubular portion and the distal closing portion, and an annular rib provided on an outer surface of the tubular portion,
the gasket pressing tool includes: a gasket pressing member having a gasket-mounting portion, which is configured to enter the lumen portion of the gasket and is made of a material harder than the gasket, and a gasket proximal surface pressing portion provided at a proximal end portion of the gasket-mounting portion; a syringe-mounting member that movably accommodates the gasket pressing member and has a proximal barrel-mounting portion that is mountable to a proximal end portion of the barrel having the flange; and a plunger that has a distal end portion entering the syringe-mounting member and is capable of pressing the gasket pressing member in a distal direction,
the gasket has an original lumen portion axial length S when the gasket is not inserted into the barrel and the gasket pressing member is not mounted, the gasket-mounting portion of the gasket pressing member has an axial length T, and the axial length T is longer than the original lumen portion axial length S, and
when the gasket is inserted into the barrel and the gasket pressing member is mounted, a mounting portion distal surface of the gasket-mounting portion of the gasket pressing member abuts on a lumen distal surface of the lumen portion of the gasket, and the gasket proximal surface pressing portion of the gasket pressing member becomes close to or abuts on a proximal surface of the gasket.

2. The liquid medicine ejection tool according to claim 1, wherein the axial length T is 1.05 to 1.20 times of the original lumen portion axial length S.

3. The liquid medicine ejection tool according to claim 1, wherein the gasket-mounting portion includes: a distal end portion having a distal surface of the mounting portion; and a proximal-side tapered portion which extends from the distal end portion of the gasket-mounting portion to the gasket proximal surface pressing portion and is enlarged in diameter toward a rear side to have a tapered shape, and a whole outer surface of the proximal-side tapered portion becomes close to or abuts on an inner surface of the lumen portion of the gasket.

4. The liquid medicine ejection tool according to claim 3, wherein a taper angle of the proximal-side tapered portion of the gasket-mounting portion is 1 to 8 degrees.

5. The liquid medicine ejection tool according to claim 1, wherein a whole outer surface of the gasket-mounting portion has a shape that substantially coincides with a whole inner surface of the lumen portion of the gasket when being inserted into the barrel.

6. The liquid medicine ejection tool according to claim 1, wherein
the lumen distal surface of the lumen portion of the gasket has: a flat central flat surface at a center of the lumen distal surface; and a lumen portion tapered surface that extends rearward and radially outward from an outer edge of the central flat surface,
the distal closing portion of the gasket has: a tapered gasket distal surface whose diameter is reduced toward a distal end; and a uniform-thickness portion which is sandwiched between the gasket distal surface and the lumen portion tapered surface and has a substantially uniform thickness,
the mounting portion distal surface of the gasket-mounting portion has: a flat distal surface, which is flat, at a center of the mounting portion distal surface; and a tapered outer edge surface extending rearward and radially outward from an outer edge of the flat distal surface, and
when the gasket is inserted into the barrel and the gasket pressing member is mounted, the flat distal surface of the gasket-mounting portion abuts on the central flat surface of the lumen portion of the gasket, and the tapered outer edge surface of the gasket-mounting portion becomes close to or abuts on the lumen portion tapered surface of the lumen portion of the gasket.

7. The liquid medicine ejection tool according to claim 1, wherein the gasket has an original axial length Q and an original lumen portion axial length S in the state where the gasket is not inserted into the syringe and the gasket pressing member is not mounted on, the gasket has a deformed axial length P and a deformed lumen portion axial length R in the state where the gasket is inserted into the syringe and the gasket pressing member is mounted, the deformed axial length P is longer than the original axial length Q, and the deformed lumen portion axial length R is longer than the original lumen portion axial length S.

8. The liquid medicine ejection tool according to claim 1, wherein the gasket pressing member includes a plunger abutment portion that is capable of abutting on and being separated from the distal end portion of the plunger.

9. The liquid medicine ejection tool according to claim 8, wherein in the state where the gasket is inserted into the barrel and the gasket pressing member is mounted, the distal end portion of the plunger abuts on the plunger abutment portion of the gasket pressing member when the plunger is advanced in the distal direction, and the mounting state of the gasket pressing member on the gasket is maintained and the plunger abutment portion is separated from the distal end portion of the plunger when the plunger is pulled in a proximal direction.

10. The liquid medicine ejection tool according to claim 9, wherein when the plunger is pulled in the proximal direction in the state where the gasket is inserted into the barrel and the gasket-mounting portion is inserted into the lumen portion of the gasket, the mounting portion distal surface of the gasket-mounting portion maintains a state of abutting on the lumen distal surface of the lumen portion of the gasket.

11. The liquid medicine ejection tool according to claim 1, wherein the syringe-mounting member includes a tubular accommodation portion that extends in the proximal direction from a body portion having the proximal barrel-mounting portion and is capable of accommodating the gasket-mounting portion.

12. The liquid medicine ejection tool according to claim 11, wherein
the plunger includes: a shaft portion which extends in the proximal direction from the distal end portion and has a shaft-side screwing portion on an outer peripheral surface; and a handle configured to rotate the plunger provided at a proximal end portion of the shaft portion,
the syringe-mounting member includes a tubular insertion portion which extends in the proximal direction from the tubular accommodation portion and in which the shaft portion of the plunger is inserted,
the insertion portion has an insertion-portion-side screwing portion, which is screwed with the shaft-side screwing portion of the shaft portion, on an inner surface, and
the plunger is advanced in the distal direction by screwing between the shaft-side screwing portion and the insertion-portion-side screwing portion along with rotation of the shaft portion.

13. A liquid medicine administration tool comprising: the liquid medicine ejection tool according to claim 1; a liquid medicine charged in the syringe; and a sealing member sealing the distal end portion of the barrel.

14. A gasket pressing tool that is mountable on a syringe, which includes: a barrel having an annular flange at a proximal end portion of the barrel; and a gasket that is slidable in the barrel and is made of an elastic material,
the gasket pressing tool comprising: a gasket pressing member configured to press the gasket; a tubular syringe-mounting member that movably accommodates the gasket pressing member and is mountable on the proximal end portion of the barrel; and a plunger which has a distal end portion entering the syringe-mounting member and is capable of pressing the gasket pressing member in a distal direction,
wherein the plunger includes: the distal end portion capable of pressing the gasket pressing member; a shaft portion that extends in a proximal direction from the distal end portion and has a shaft-side screwing portion on an outer peripheral surface; and a handle configured to rotate the plunger provided at a proximal end portion of the shaft portion,
the syringe-mounting member includes: a body portion capable of accommodating the flange of the barrel; a tubular accommodation portion which extends from a proximal end of the body portion in a proximal direction of the gasket pressing tool and is capable of accommodating the gasket pressing member; and a tubular insertion portion which extends from a proximal end of the tubular accommodation portion in the proximal direction of the gasket pressing tool and in which the shaft portion of the plunger is inserted,
the insertion portion of the syringe-mounting member has an insertion-portion-side screwing portion, which is screwed with the shaft-side screwing portion of the shaft portion, on an inner peripheral surface,
the body portion of the syringe-mounting member includes: a distal-side support portion capable of supporting a distal surface of the flange; a proximal-side support portion capable of supporting a proximal surface of the flange; a connecting portion connecting the distal-side support portion and the proximal-side support portion; a flange accommodation portion which is formed of the distal-side support portion, the proximal-side support portion, and the connecting portion, and is capable of accommodating the flange; and a flange insertion opening which is open to a lateral side of the body portion and is configured to insert the flange into the flange accommodation portion, and
the distal-side support portion has an axial thickness of 5 to 25 mm along a central axis of the syringe-mounting member, the proximal-side support portion has an axial thickness of 5 to 25 mm along the central axis of the syringe-mounting member, and the connecting portion has a radial width of 1 to 5 mm along a radial direction of the syringe-mounting member.

15. A liquid medicine administration tool comprising: the gasket pressing tool according to claim 14; the syringe; a liquid medicine charged in the syringe; and a sealing member sealing the distal end portion of the barrel.

16. A gasket pressing tool for a syringe, which is mountable on a syringe having a barrel and a gasket slidable in the barrel,
the gasket pressing tool comprising: a gasket pressing assembly which includes a gasket pressing member and a sound generating member; a syringe-mounting member which has a syringe-mounting portion and movably accommodates the gasket pressing assembly; and a plunger which enters the syringe-mounting member on a distal side and is capable of pressing the gasket pressing assembly by rotation,
wherein the sound generating member includes a claw portion which is elastically deformable, the gasket pressing member includes a wall portion abutting on the claw portion, and the claw portion includes a claw tip portion protruding from the wall portion,
the plunger includes a claw-flicking rib provided on a side surface of the distal end portion,
the claw-flicking rib is located on a rear side of the claw tip portion in a state where the plunger does not abut on the gasket pressing assembly in a pressable manner, and the claw-flicking rib is located on a lateral side of the claw tip portion in a state where the plunger abuts on the gasket pressing assembly in a pressable manner, and as the plunger is rotated, the plunger advances to move the gasket pressing assembly forward, the claw-flicking rib abuts on the claw tip portion and passes the claw portion while deforming the claw portion, and a sound is generated as the claw portion restored after the passage abuts on the wall portion.

17. A liquid medicine administration tool comprising: the gasket pressing tool for a syringe according to claim 16; and a prefilled syringe that includes a barrel, a liquid medicine charged in the barrel, and the gasket slidably accommodated in the barrel, wherein the prefilled syringe is mounted on the syringe-mounting portion of the gasket pressing tool for a syringe.
